(19) United States
(12) Patent Application Publication
(10) Pub. No.: US 2026/0176685 A1
(43) Pub. Date: Jun. 25, 2026

(54) PREDICTING CANCER CELL EXPRESSION BY ANALYZING METHYLATION STATUS OF CTDNA

(71) Applicant: Foundation Medicine, Inc., Cambridge, MA (US)

(72) Inventors: Brian Giacopelli, Medford, MA (US); Alex Robertson, Princeton, NJ (US); Neil Peterman, San Francisco, CA (US)

(73) Assignee: Foundation Medicine, Inc., Cambridge, MA (US)

(21) Appl. No.: 19/420,569

(22) Filed: Dec. 15, 2025

Related U.S. Application Data
(63) Continuation of application No. PCT/US2024/034123, filed on Jun. 14, 2024.
(60) Provisional application No. 63/508,371, filed on Jun. 15, 2023.

(51) Int. Cl. C12Q 1/6869; C12Q 1/6886

(52) U.S. Cl. CPC C12Q 1/6869; C12Q 1/6886; C12Q 2600/154

(57) ABSTRACT

Techniques for predicting expression of cancer cells based on the methylation status of a region of DNA are described. An example method includes identifying data indicative of cell free DNA (cfDNA) from a sample derived from a subject. A methylation status of one or more regions of circulating tumor DNA (ctDNA) among the cfDNA is identified by analyzing the data. The example method further includes inputting input data including the methylation status of the one or more regions into at least one model configured to generate a probability that cancer cells of the subject express a predetermined sequence. In addition, the example method includes generating a report based on the probability that the cancer cells of the subject express the predetermined sequence.

### CROSS-REFERENCE TO RELATED APPLICATIONS

This application is a continuation application of International App. No. PCT/US2024/034123, filed on Jun. 14, 2024, which claims the priority of U.S. Provisional App. No. 63/508,371, filed on Jun. 15, 2023, each of which is incorporated by reference herein in its entirety.

### BACKGROUND

Many cancers arise based on genetic and/or epigenetic changes that result in unregulated cell division. Oncogenic transformation is inextricably linked to cancer-specific patterns of gene expression, and different types or subtypes of cancer have divergent patterns of aberrant gene expression. Further, cancer cells may express different genes than other cells in the body. Many promising anticancer therapies target cells that express specific genes. However, it can be difficult to determine whether cancer cells express genes that make them responsive to these therapies. For example, whole transcriptome sequencing can be performed to determine what types of genes are being expressed by a cancer cell. However, whole transcriptome sequencing can be costly and inconvenient for patients.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 illustrates an example environment for predicting cancer cell expression by analyzing the methylation status of cell-free DNA (cfDNA).

FIG. 2 illustrates an example environment illustrating circulating tumor DNA (ctDNA), which can be utilized to analyze cancer cells of a subject.

FIG. 3 illustrates an example environment for training and utilizing a predictive model 302 to determine expression of cancer cells based on methylation statuses of regions of DNA derived from the cancer cells.

FIG. 4 illustrates an example of training data utilized to train one or more ML models.

FIG. 5 illustrates an example report summarizing predicted categories of a cancer of a subject.

FIG. 6 illustrates an example process for determining a methylation status of a sample.

FIG. 7 illustrates an example process for recommending an anticancer treatment based on a methylation status of a sample.

FIG. 8 illustrates an example environment for sequencing various nucleic acid molecules.

FIG. 9 illustrates one or more devices configured to perform various operations described herein.

FIG. 10 illustrates an example process utilized in an Experimental Example, described below.

FIG. 11 illustrates example results of an analysis performed on regions of ctDNA related to the MAPK signaling pathway.

Some of the drawings submitted herewith may be better understood in color. Applicant considers the color versions of the drawings as part of the original submission and reserves the right to present color images of the drawings in later proceedings.

### DETAILED DESCRIPTION

Various implementations of the present disclosure relate to techniques for evaluating expression of cancer cells by analyzing a methylation status of cancer cell DNA. In particular cases, the methylation status is determined based on circulating tumor DNA (ctDNA). In some examples, ctDNA can be extracted from a fluid biopsy sample (e.g., a serum sample). Thus, according to various implementations described herein, the subject's cancer (or tumor) can be analyzed and categorized expeditiously using a minimally invasive liquid biopsy procedure and without performing RNA sequencing.

Implementations of the present disclosure provide significant improvements to the technical field of cancer diagnosis, management and treatment. Using previous technologies, a patient's tumor could be analyzed by performing a tissue biopsy on a potential tumor and also performing histological staining and additional analysis on the tissue biopsy sample. This process is problematic in several respects. For instance, a tissue biopsy can be dangerous and/or uncomfortable for the patient. Scheduling tissue biopsies can be challenging, because they generally involve the efforts of surgeons, anesthesiologists, and other medical staff in specialized surgical settings. After a tissue biopsy sample is obtained, it can take an extended period of time (e.g., weeks) to be stained and examined by a pathologist, which can delay care and cause significant emotional hardship for the subject. Further, histological staining procedures performed in many clinical environments are nevertheless unable to differentiate between some types of cancers, such that the process may result in erroneous or inconclusive classification. In contrast, implementations of the present disclosure can utilize samples obtained intravenously or through other minimally invasive means. Further, analyses described herein can be performed rapidly and with high accuracy.

Various analyses described herein cannot be performed in the human mind, or by pen and paper. For example, a sample obtained from a subject may contain numerous (e.g., millions) of cfDNA fragments to be analyzed. In various cases, it would be impossible to manually or mentally identify which of the cfDNA are ctDNA and which are non-ctDNA. Further, it would be impossible to manually or mentally identify relevant methylation statuses based on the ctDNA. In addition, it would be impossible to manually or mentally attribute methylation statuses that are relevant to the expression of the cancer cells from which the ctDNA originated. Particular implementations of the present disclosure are fundamentally tied to computer technology, and do not represent mere automation of processes that are performed manually.

### Example Definitions

As used herein, the terms “deoxyribonucleic acid,” “DNA,” “DNA molecule,” and their equivalents, may refer to a polymer of nucleotides (also referred to as “nucleobases”) containing deoxyribose. The nucleotides in DNA include cytosine (C), guanine (G), adenine (A), and thymine (T). Each DNA nucleotide includes a deoxyribose and a phosphate group. An example single-stranded DNA (ssDNA) molecule includes a chain of covalently bonded DNA nucleotides. In the example ssDNA molecule, the phosphate group of the mth nucleotide is covalently bonded to the deoxyribose of the (m−1)th nucleotide, wherein m is a positive integer greater than 2 and less than or equal to the number of DNA nucleotides in the chain. In various examples, DNA is double-stranded and includes two ssDNA molecules that are complementary to one another and coiled around each other in a double helix form. The nucleotides of one ssDNA molecule are hydrogen bonded to the nucleotides of the other ssDNA molecule. In particular, the pyrimidines (A and T) hydrogen bond to each other, and the purines (C and G) hydrogen bond to each other.

As used herein, the terms “ribonucleic acid,” “RNA,” “RNA molecule,” and their equivalents, may refer to a polymer of nucleotides containing ribose. The nucleotides in RNA include cytosine (C), guanine (G), adenine (A), and uracil (U). Each RNA nucleotide includes a ribose and a phosphate group. In an example RNA molecule, the phosphate group of the nth nucleotide is covalently bonded to the ribose of the (n−1)th nucleotide, wherein n is a positive integer greater than 2 and less than or equal to the number of RNA nucleotides in the chain. Messenger RNA (mRNA) is a type of RNA molecule that is synthesized (or “transcribed”) by RNA polymerase (an enzyme) to be complementary to a gene encoded in a DNA sequence, and is also used by a ribosome to synthesize a polypeptide or protein. An mRNA is therefore an example of a “coding RNA.” In various cases, intron sequences are removed from an mRNA via a process known as “RNA splicing.” MicroRNA (“miRNA”) are single-stranded RNA molecules that perform post-transcriptional gene expression regulation. For instance, a miRNA may bind to a complementary mRNA molecule, thereby cleaving, destabilizing, or otherwise preventing the mRNA molecule from being translated into a polypeptide or protein by a ribosome. In various examples, a miRNA has a length in a range of 21 to 23 RNA nucleotides. As used herein, the terms “non-coding RNA” may refer to a type of RNA that is not translated into a protein. Examples of non-coding RNA include miRNA, transfer RNA (tRNA), and ribosomal RNA (rRNA). The term “functional RNA,” and its equivalents, may refer to any RNA molecule that impacts a biological process. For instance, functional RNA may include mRNA, miRNA, tRNA, IRNA, and the like.

As used herein, the term “base,” and its equivalents, may refer to a monomer of a polymer. For example, a base of DNA or RNA is a nucleotide.

As used herein, the term “base pair,” and its equivalents, may refer to a pair of complementary DNA nucleotides, which are hydrogen-bonded to one another in a double-stranded DNA molecule. For example, a base pair includes a first base in a first ssDNA and a second base in a second ssDNA, wherein the first and second bases are complementary and hydrogen-bonded to one another.

As used herein, the terms “nucleotide,” “nucleobase,” “nucleic acid,” “nucleic acid molecule,” and their equivalents, may refer to an organic molecule that includes a nitrogenous base, a sugar, and a phosphate group. In various cases, a nucleotide is a monomer of DNA or RNA. A nucleotide, for instance, is a chemical structure.

As used herein, the terms “3′ end,” “3-prime end,” and their equivalents, may refer to a terminus of a single-stranded nucleotide polymer that includes a base whose third carbon in its deoxyribose or ribose is bound to a hydroxyl group while being unbound to another base.

As used herein, the terms “5′ end,” “5-prime end,” and their equivalents, may refer to a terminus of a single-stranded nucleotide polymer that includes a base whose fifth carbon in its deoxyribose or ribose ring is unbound to another base. In some cases, the fifth carbon is bound to a phosphate group.

As used herein, the “length” of a polymer refers to a number of covalently bonded monomers that are included in the polymer. For instance, the length of a DNA molecule may be the number of covalently bonded nucleotides in at least one strand of the DNA molecule and/or the number of base pairs in the DNA molecule. In various examples, the length of an RNA molecule may be the number of covalently bonded nucleotides in the RNA molecule.

As used herein, the term “gene,” and its equivalents, refers to a sequence of DNA nucleotides that is transcribed into a functional RNA. The functional RNA, for instance, is RNA that is translated into a polypeptide or protein (e.g., mRNA) or that has some other biological function (e.g., miRNA, tRNA, etc.). A gene is “expressed” when it is used as a template to generate a functional RNA. A subject, for instance, has numerous genes contained in the subject's genome. A gene may include both introns and exons. As used herein, the term “intron,” and its equivalents, may refer to a subset of DNA nucleotides in a gene that is not used to code for any functional RNA that is expressed by the organism. As used herein, the term “exon,” and its equivalents, may refer to a subset of DNA nucleotides in a gene that is used to code for a functional RNA. For instance, an exon may encode a polypeptide or protein that is expressed by the organism. In various examples, a gene can be represented in data (e.g., as data representative of the sequence of DNA nucleotides in the gene) or as a chemical structure (e.g., as the sequence of DNA nucleotides itself).

As used herein, the term “genome,” and its equivalents, refers to the aggregate of genes of a subject. In various cases, a genome represents the sequences of several linear DNA molecules that are present in a subject's chromosomes. A “reference genome” refers to an aggregation of genes of one or more reference subjects. In various cases, a genome is represented in data.

As used herein, the terms “pangenome,” “pan-genome,” “supragenome,” and their equivalents, refers to an aggregate set of genes from multiple subgroups (e.g., strains) within a population (e.g., a clade) of subjects. A pangenome, for example, indicates genes that are present in all subjects within the population, as well as genes that are present in some of the subjects of the population. A pangenome is represented in data, for instance.

As used herein, the term “transcriptome,” and its equivalents, refers to the aggregate of RNA sequences of a subject. In some cases, a transcriptome is limited to mRNA sequences. In various examples, a transcriptome is represented in data.

As used herein, the term “genomic DNA,” “gDNA,” “chromosomal DNA,” and their equivalents, may refer to DNA molecules that are obtained from a chromosome and/or nucleus of a cell.

As used herein, the terms “DNA fragment,” “fragment,” and their equivalents, may refer to DNA molecules that are excised and/or broken off from a larger DNA molecule.

As used herein, the terms “cell-free DNA,” “cfDNA,” and their equivalents, may refer to DNA fragments that are non-encapsulated and obtained outside of cells within a sample (e.g., a liquid biopsy sample).

As used herein, the terms “circulating tumor DNA,” “ctDNA,” and their equivalents, may refer to a cfDNA molecule that originates from a cancer cell.

As used herein, the terms “end motif,” “terminal sequences,” and their equivalents, may refer to a sequence of nucleotides extending from a 3′ or 5′ end of a DNA or RNA molecule. In various cases, the end motif is shorter than a length of the DNA or RNA molecule. For example, the end motif may have a length in a range of 5 to 30 bases or base pairs, a range of 3 to 30 bases or base pairs, or a range of 1 to 30 base pairs.

As used herein, the term “promoter,” and its equivalents, may refer to a portion of a DNA molecule that binds one or more proteins in order to initiate transcription of a gene. For example, the promotor is located “upstream” of the gene. For example, the promotor is located between the 5′ end of the DNA molecule and the gene. A promotor may include one or more binding sites for RNA polymerase, and/or one or more transcription factor binding sites. In some examples, a promotor includes one or more CpG islands. A promoter, for instance, includes a transcription start site.

As used herein, the terms “CpG island,” “CGI,” “CpG site,” and their equivalents, may refer to a continuous portion of a DNA molecule whose sequence includes greater than a threshold amount (e.g., greater than 50%) of G-C base pairs. As used herein, the term “enhancer,” and its equivalents, may refer to a portion of a DNA molecule that binds one or more proteins in order to increase the chance that a gene will be transcribed. For instance, an enhancer includes one or more transcription factor binding sites. In various cases, an enhancer includes one or more CpG islands.

As used herein, the terms “DNA methylation,” “methylation,” and their equivalents, may refer to a process by which methyl groups are added to cytosines of a DNA molecule. The presence of the methyl groups can regulate the expression of nearby (e.g., within a threshold number of base pairs) genes within the DNA molecule by preventing molecules from binding to the portion of the DNA molecule that is methylated. For instance, if many cytosines are methylated in a CpG island present in a promoter, the methyl groups may prevent the attachment of RNA polymerase to the promoter, thereby preventing the gene associated with the promoter from being transcribed and expressed.

As used herein, the term “cancer,” and its equivalents, may refer to a condition of a subject in which particular cells (referred to as “cancer cells”) divide uncontrollably in the subject's body. In some cases, a cancer is characterized by a location or tissue type from which the cancer cells originated. In some examples, a cancer is characterized by a location or tissue type in which the cancer cells are located.

As used herein, the terms “tumor,” “neoplasm,” and their equivalents, may refer to a mass of tissue including cancer cells.

As used herein, the terms “tissue of origin,” “tissue origin,” and their equivalents, refers to a differentiated type of tissue from which cancer cells in the body of a subject began dividing uncontrollably in the subject's body.

As used herein, the terms “liquid biopsy,” “fluid biopsy,” and their equivalents, may refer to a process of obtaining a fluid sample from a subject's body. The sample, for instance, can be referred to as a “liquid biopsy sample.” Examples of fluids that are sampled from the body include blood, plasma, cerebrospinal fluid, sputum, stool, urine, lymphatic fluid, and saliva.

As used herein, the term “tissue biopsy,” and its equivalents, may refer to a process of obtaining a sample of cells from a subject's body. A tissue biopsy, in various cases, is performed by cutting a mass of cells from the subject's body. For instance, a tissue biopsy is a procedure performed by a surgeon, interventional radiologist, interventional cardiologist, or other specialized clinician. The term “tissue” or “tissue biopsy sample” can be used to refer to the sample of cells obtained using a tissue biopsy.

As used herein, the term “subject,” and its equivalents, may refer to a human or non-human animal. A subject that is receiving care from at least one care provider may be referred to as a “patient.”

As used herein, the terms “machine learning,” “ML,” “computer learning,” “artificial intelligence,” and their equivalents, may refer to the use of a computing devices to learn patterns in training data. The process of learning these patterns may be referred to as “training.” In particular cases, one or more computing devices may perform machine learning by executing a machine learning model. As used herein, the terms “machine learning model,” “ML model,” and their equivalents, may refer to data encoding instructions that, when executed by at least one computing device, causes the at least one computing device to learn patterns in training data by optimizing one or more metrics, values, or other types of parameters. After training, an ML model, when executed by at least one computing device, causes the at least one computing device to utilize the optimized parameters in order to perform one or more tasks.

As used herein, the term “variant,” and its equivalents, may refer to a difference between a subject genetic sequence and a reference sequence. For instance, a variant may correspond to a difference between one or more nucleotides in a genome of a subject and one or more corresponding nucleotides in at least one reference genome or pangenome. A variant may be characterized by its identity (e.g., what nucleotides are different), its position (e.g., where are the nucleotides located in the genome, what chromosome contains the nucleotides, what gene contains the nucleotides, etc.), its length (e.g., how many nucleotides are different from the reference sequence), its type (e.g., substitution, insertion, deletion, copy number alternation, rearrangement of fusion, etc.), and other features that indicates its significance and/or relevance. In some cases, a variant represents any apparent alteration in a sequence that has been read from a nucleic acid molecule with respect to the reference sequence, such as restriction enzyme (RE) reads. In various examples, a variant can be represented in data (e.g., by data characterizing the variant) or as a chemical structure (e.g., the nucleotides themselves). As used herein, the term “mutation,” and its equivalents, may refer to a change in a gene.

As used herein, the term “substitution,” and its equivalents, can refer to a nucleotide in a subject sequence that is different than an equivalent nucleotide (e.g., a nucleotide at the same position) in a reference sequence.

As used herein, the term “insertion,” and its equivalents, can refer to a nucleotide in a subject sequence that is added with respect to a reference sequence.

As used herein, the term “deletion,” and its equivalents, can refer to the removal of a nucleotide from a nucleotide sequence.

As used herein, the terms “copy number alternation,” “CNA,” “copy number variation,” “CNV,” and their equivalents, can refer to a portion of a reference sequence that is repeated.

As used herein, the terms “rearrangement of fusion,” “fusion rearrangement,” “translocation,” and their equivalents, can refer to a change in the relative position of one or more portions of a reference sequence, thereby generating a gene that was not present in the reference sequence.

As used herein, the term “sequencing,” and its equivalents, may refer to a process of identifying the order and identity of monomers in a polymer chain, such as the order and identity of nucleotides in a DNA or RNA molecule. The terms “whole genome sequencing,” “WGS,” and their equivalents, may refer to the process of sequencing an entire genome of a subject, including the introns and exons of the genes of the subject. The term “whole exome sequencing,” and its equivalents, may refer to the process of sequencing all exomes of a subject. The term “targeted sequencing,” and its equivalents, may refer to the process of sequencing a portion of the genome of a subject, such as sequencing a single gene of the subject. Various techniques can be utilized to sequence a DNA or RNA molecule, such as massively parallel sequencing (MPS), nanopore sequencing, direct sequencing, Sanger sequencing, or next-generation sequencing. In various cases, sequencing is performed on physical molecules (e.g., RNA or DNA) and is used to generate data.

As used herein, the terms “massive parallel sequencing,” “massively parallel sequencing,” “MPS,” and their equivalents, may refer to a technique for simultaneously performing multiple reactions that can be used to identify the order and identity of monomers in multiple polymer chains. In particular cases, massive parallel sequencing can be performed using sequencing-by-synthesis on clonally amplified DNA molecules that are located in spatially separated regions, which are individually monitored by sensors.

As used herein, the term “nanopore sequencing,” and its equivalents, may refer to a technique for identifying the order and identity of monomers in a polymer chain by transporting the polymer chain from a first space to a second space, wherein the first space and the second space are separated by a substrate, by directing the polymer chain through a small hole (known as a “nanopore”) embedded in the substrate, and monitoring a relative electrical signal (e.g., a voltage or current) between the first space and the second space.

As used herein, the term “sensor,” and its equivalents, may refer to a physical device or other apparatus that is configured to detect one or more detection signals.

As used herein, the term “detection signal,” and its equivalents, may refer to a physical signal that can be identified, characterized, or otherwise perceived by a sensor.

As used herein, the term “sequence read data,” and its equivalents, may refer to data that is indicative of an order and identity of monomers in a polymer, such as the order and identity of nucleotides in a DNA or RNA sequence. In various implementations, sequence read data is generated via a sequencing operation.

As used herein, the term “image,” and its equivalents, may refer to 2D or 3D array of data indicative of an array of pixels or voxels.

As used herein, the term “ligating,” and its equivalents, may refer to a process of joining two molecules together, for example, with a chemical bond.

As used herein, the term “adapter,” and its equivalents, may refer to an oligonucleotide that can be ligated to a target nucleic acid molecule. In various cases, an adapter prepares the target nucleic acid molecule for sequencing.

As used herein, the term “bait molecule,” and its equivalents, may refer to a nucleic acid molecule having a region that is complementary to a region of a target molecule (e.g., cfDNA). A bait molecule includes, for instance, a nucleic acid molecule that can hybridize to (i.e., is complementary to) a target molecule can be used to capture the target molecule. In some instances, the bait molecule is a capture oligonucleotide (or capture probe). In some instances, the bait molecule is suitable for solution phase hybridization to the target molecule. In some instances, the bait molecule is suitable for solid phase hybridization to the target molecule. In some instances, the bait molecule is suitable for both solution-phase and solid-phase hybridization to the target molecule. The design and construction of bait molecules is described in more detail in, e.g., International Patent Application Publication No. WO 2020/236941.

As used herein, the term “amplifying,” and its equivalents, may refer to a process of generating copies of a target molecule, such as a nucleic acid molecule.

As used herein, the term “hybridization,” and its equivalents, may refer to a process by which to complementary single-stranded nucleic acid molecules bind to one another, thereby forming a double-stranded nucleic acid molecule. In certain examples, the double-stranded nature of the nucleic acid molecule is maintained under stringent hybridization conditions. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50° C.

As used herein, the term “complementary,” and its equivalents, may refer to a state of two single-stranded nucleic acid molecules with respective sequences that cause the nucleic acid molecules to spontaneously hybridize to one another. One nucleic acid molecule, for instance, may have a sequence that causes each nucleic acid to hydrogen bond to a respective nucleic acid in the other nucleic acid molecule.

As used herein, the terms “therapy,” “treatment,” and their equivalents, may refer to a composition or process that can be used to remediate a health problem. Cancer therapies, for instance, include surgery, radiotherapy, chemotherapy, immunotherapy, cell-based therapies, and the like. Examples of cancer therapies include abemaciclib (Verzenio), abiraterone acetate (Zytiga), acalabrutinib (Calquence), ado-trastuzumab emtansine (Kadcyla), afatinib dimaleate (Gilotrif), aldesleukin (Proleukin), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), copanlisib hydrochloride (Aliqopa), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), iobenguane 1131 (Azedra), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), Lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximabcmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), panobinostat (Farydak), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecanhziy (Trodelvy), seliciclib, selinexor (Xpovio), selpercatinib (Retevmo), selumetinib sulfate (Koselugo), siltuximab (Sylvant), sipuleucel-T (Provenge), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), and combinations thereof. Examples of cancer therapies also include targeted antibody-based therapies (antibody-drug conjugates, antibody-radioisotope conjugates, and targeted immune cell therapies (e.g., immune effector cells genetically modified to express a chimeric antigen receptor (CAR).

As used herein, the term “treatment-responsive,” and its equivalents, may refer to a type of cancer cells that can be substantially killed using a predetermined type of therapy.

As used herein, the term “treatment-resistant,” and its equivalents, may refer to a type of cancer that cannot be substantially killed using a predetermined type of therapy.

As used herein, the term “metastasis profile,” and its equivalents, may refer to a propensity of a type of cancer to metastasize into one or more differentiated tumor types besides the cancer's tissue origin. In some implementations, the metastasis profile can further indicate the type of tissue in which the cancer can or is likely to metastasize.

As used herein, the term “clinical trial,” and its equivalents, may refer to a research study used to evaluate a hypothesis based on participation by one or more subjects. In various examples, a clinical trial can be used to assess the efficacy and/or safety of a proposed therapy. A clinical trial may be performed in furtherance of approval of a treatment by a regulatory authority (e.g., the United States Food & Drug Administration (FDA).

### DESCRIPTION OF EXAMPLE IMPLEMENTATIONS

Various implementations of the present disclosure will now be described with reference to the accompanying Figures.

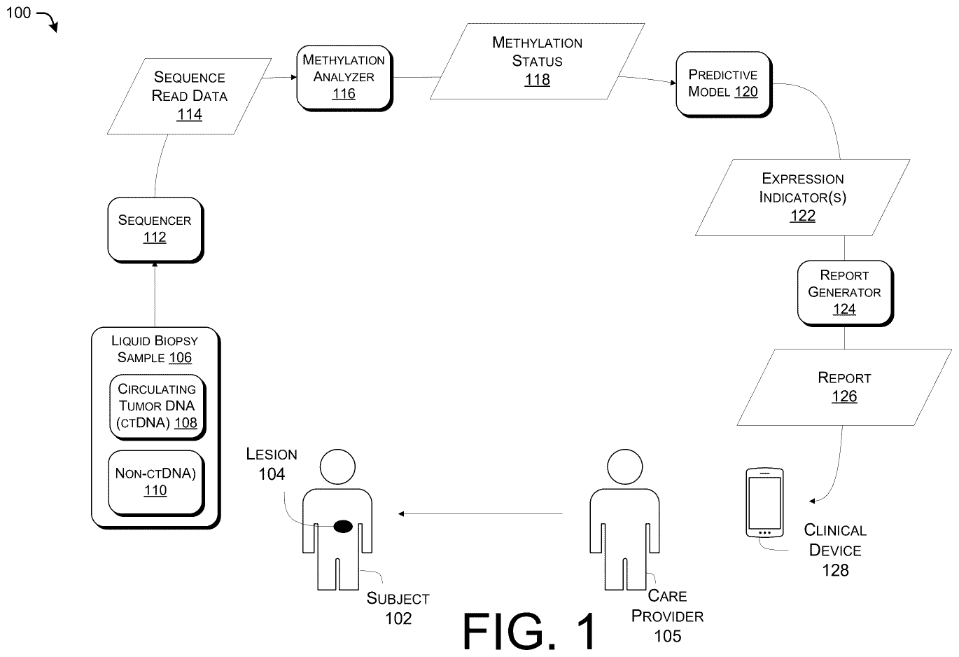

FIG. 1 illustrates an example environment 100 for predicting cancer cell expression by analyzing the methylation status of cell-free DNA (cfDNA). A subject 102, for instance, may present to a clinical environment with a lesion 104. In various cases, the lesion 104 may be a tumor that includes cancer cells. According to various examples, the subject 102 has one or more types of cancer, such as adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease, intestinal cancer, kidney cancer, larynx cancer, leukemia, liver cancer, lymph node cancer, lymphoma, lung cancer, melanoma, mesothelioma, myeloma, nasopharynx cancer, a neuroblastoma, non-Hodgkin's lymphoma, oral cancer, ovarian cancer, pancreatic cancer, penile cancer, pharynx cancer, prostate cancer, rectal cancer, sarcoma, seminoma, skin cancer, stomach cancer, a teratoma, testicular cancer, thyroid cancer, uterine cancer, vaginal cancer, a vascular tumor, or combinations or metastases thereof.

In some embodiments, the subject 102 has a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

In some embodiments, the subject 102 has acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), a non-small cell lung cancer KRAS (+/−G12C), a non-small cell lung cancer TMB-H, a non-small cell lung cancer MET exon 14 skipping, a non-small cell lung cancer ERBB2 inframe indel, a non-small cell lung cancer EGFR exon 20 indel, ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In various cases, a care provider 105 is responsible for diagnosing and/or treating the subject 102. According to some implementations, the lesion 104 may be initially identified using a noninvasive technique. For example, the lesion 104 may be visualized using an imaging modality, such as ultrasound, x-ray, computed tomography (CT), magnetic resonance imaging (MRI), positron emission tomography (PET), single photon emission CT (SPECT), or any combination thereof. Using the noninvasive technique, the care provider 105 may identify the presence of the lesion 104, but may be unable to determine whether the lesion 104 is a cancerous tumor using noninvasive diagnostic methodologies. In some cases in which the lesion 104 is a tumor, the care provider 105 may be unable to identify whether the tumor is metastatic or benign. In some examples, the care provider 105 is unable to determine a therapy for treating the tumor effectively. For instance, the types of genes expressed by a cancer cell are relevant to whether the cancer cell is responsive or resistant to a particular treatment.

The care provider 105 could determine whether the lesion 104 was treatable by a particular anticancer therapy by initiating a tissue biopsy on the subject 102. For instance, the care provider 105 could surgically remove a tissue sample from the lesion 104 and/or review the tissue sample using histochemistry and/or immunohistochemistry in order to classify the lesion 104. Tumor classifications, for instance, can be indicative of responsiveness to anticancer therapies. However, attempting to classify the lesion 104 using tissue biopsy has several drawbacks. First, the tissue biopsy could be a highly invasive surgical procedure, which can cause significant discomfort to the subject 102. Second, the tissue biopsy may require the subject 102 to undergo general anesthesia, which could be dangerous to the subject 102. Third, even if the tissue biopsy was performed and a tissue sample was obtained, it may not be classifiable using conventional histological techniques, such as conventional immunohistochemical staining and review. Fourth, it is unlikely that the single care provider 105 would be trained to perform the tissue biopsy (which would be performed by a surgeon), to administer anesthesia to the subject 102 during the tissue biopsy (which would be performed by an anesthesiologist), and the analysis of the tissue biopsy (which would be performed by a trained pathologist), such that the classification would utilize multiple highly trained care providers. Even if the cells in the lesion 104 could be analyzed by these means, the coordinated efforts of these care providers could delay diagnosis and treatment of the lesion 104, and could cause significant expense to the subject 102. In various examples, the delay in diagnosis and treatment could cause significant emotional hardship to the subject 102. Further, the delay in diagnosis and treatment could delay a therapy of the lesion 104, which could cause lasting harm to the subject 102, particularly in cases in which the lesion 104 is representative of an aggressive form of cancer. Notably, if the subject 102 is located in a low-resource setting or rural clinical environment, the subject 102 may be unable to participate in the tissue biopsy without traveling to a clinical environment that is capable of performing and analyzing the tissue biopsy, causing further delays and disruptions.

In various implementations, the lesion 104 is classified without requiring a tissue biopsy. For instance, a liquid biopsy sample 106 is obtained from the subject 102. The liquid biopsy sample 106, for instance, includes pleural lavage, blood, plasma, cerebrospinal fluid, sputum, stool, urine, lymphatic fluid, saliva, or some other fluid obtained from the body of the subject 102. In some cases, a blood sample is obtained intravenously from the subject 102. The liquid biopsy sample 106, according to various examples, is a plasma sample obtained from the blood of the subject 102. The liquid biopsy sample 106 can be obtained in a minimally invasive procedure, which could be performed by a medical technician rather than a surgeon.

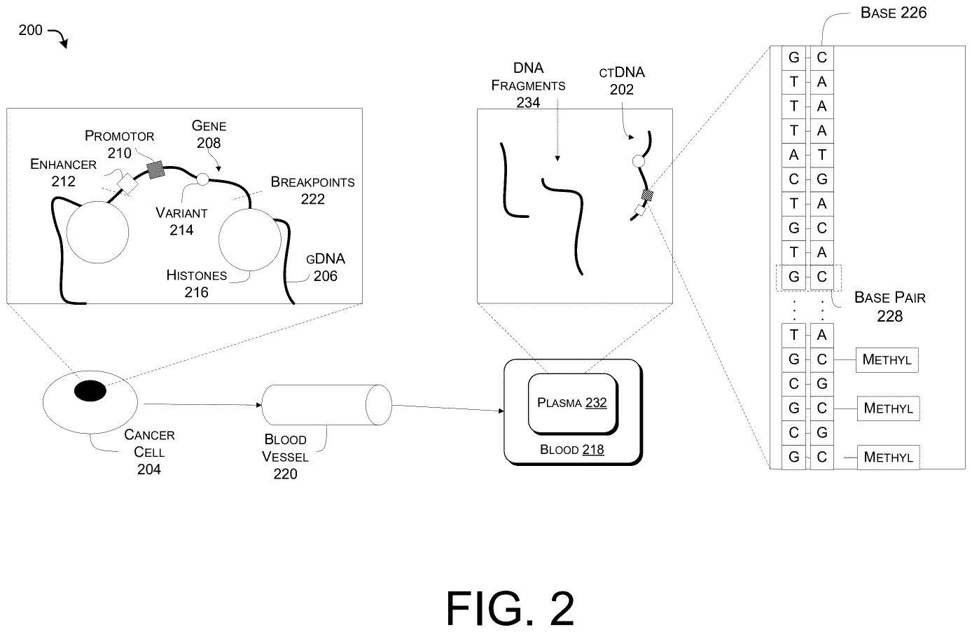

The liquid biopsy sample 106 includes nucleic acid molecules in the form of cfDNA. In examples in which the subject 102 has cancer (e.g., the lesion 104 is a cancerous tumor), the cfDNA, for instance, includes circulating tumor DNA (ctDNA) 108 as well as non-ctDNA 110. In cases wherein the lesion 104 is a tumor, cancer cells within the lesion 104 will lyse and release the ctDNA 108 into the bloodstream of the subject 102. Further, other cells additionally release non-ctDNA into the bloodstream of the subject 102. In general, the cfDNA includes fragments with lengths that are in a range of 1 to 500, 3 to 500, or 100 to 500 bases long. For instance, the cfDNA includes fragments that are about 170 bases long and/or fragments that are about 340 bases long. For example, the cfDNA includes fragments that are 100 to 240 bases long and/or fragments that are 270 to 410 bases long. As will be described in further detail with respect to FIG. 2, the features of the ctDNA 108 are indicative of the expression of the cancer cells within the lesion 104. That is, the features of the ctDNA 108 may be indicative of one or more genes that are expressed by the cancer cells.

In various cases, the liquid biopsy sample 106 is transported to a location that is remote from the subject 102 for further processing. For example, the liquid biopsy sample 106 is removed from the subject 102 in a clinical environment (e.g., a hospital) and is then transported to a remote laboratory for further testing and analysis.

A sequencer 112 is configured to generate sequence read data 114 indicating the sequences of the ctDNA 108 and, optionally, the non-ctDNA 110. Non-ctDNA sequencing is considered optional, for example, when pre-analytical means to enrich for the ctDNA component of cfDNA are used to generate the sequencing library (e.g., oversampling of shorter cfDNA fragments for inclusion in the sequencing library). The sequencer 112, for instance, includes one or more devices that are configured to generate the sequence read data 114 by processing at least a portion of the liquid biopsy sample 106. In some cases, the cfDNA including the ctDNA 108 and the non-ctDNA 110 is extracted from the liquid biopsy sample 106. The extraction can be performed by the sequencer 112, by another device, manually (e.g., by a laboratory technician), or any combination thereof. Any appropriate extraction method known to those of ordinary skill in the art can be utilized.

In various cases, the sequencer 112 is configured to perform one or more processes (e.g., chemical reactions) on the cfDNA in order to prepare the cfDNA for sequencing. For instance, the sequencer 112 may ligate adapters onto the cfDNA and/or amplify the cfDNA, such that numerous copies of the ligated cfDNA are available for sequencing. Examples of the adapters include, for example, amplification primers, flow cell adapter sequences, substrate adapter sequences, or sample index sequences. The cfDNA (e.g., the ligated cfDNA) may be amplified by generating multiple copies of the cfDNA using one or more techniques such as polymerase chain reaction (PCR), a non-PCR amplification technique, or an isothermal amplification technique.

The sequencer 112 may identify the length, position, and identity of the bases in the cfDNA by sequencing the cfDNA (e.g., the amplified and/or ligated cfDNA). In various implementations, the sequencer 112 utilizes first-generation sequencing (e.g., Sanger sequencing), second-generation sequencing (e.g., massive parallel sequencing), third-generation sequencing (e.g., nanopore sequencing), or a combination thereof. In some cases, the sequencer 112 is configured to sequence substantially all of the nucleotides of all of the cfDNA fragments obtained from the liquid biopsy sample 106. In some examples, the sequencer 112 is configured to perform targeted sequencing. For instance, the sequencer 112 may determine whether the cfDNA fragments contain one or more predetermined sequences.

In various cases, the sequencer 112 includes one or more sensors that are configured to detect physical signals (also referred to as “detection signals”) that are indicative of the nucleotide sequences of the cfDNA fragments. The sequencer 112 may perform sequencing-by-synthesis. For example, the sequencer 112 may include one or more optical sensors configured to detect optical signals emitted from fluorescently tagged dNTPs that are joined together in a synthesized DNA strand using the ligated cfDNA as templates. The optical signals detected by the optical sensor(s), for instance, are indicative of the sequences of the cfDNA. The sequencer 112 may perform nanopore sequencing. In various cases, the sequencer 112 includes one or more electrical sensors configured to measure an electrical signal (e.g., an electrical current) across a substrate as the ligated cfDNA fragments are directed through a nanopore extending through the substrate. The electrical signal over time, in various cases, is indicative of the sequences of the cfDNA in the liquid biopsy sample 106. The sequencer 112, in various implementations, is configured to generate the sequence read data 114 as digital data based on the analog signals detected by the sensor(s). For instance, the sequencer 112 includes one or more analog to digital converters (ADCs). In various cases, the sequencer 112 includes at least one processor configured to generate the sequence read data 114.

In some examples, the sequencer 112 performs methylation sequencing. For example, the sequencer 112 may expose the cfDNA to a reagent (e.g., including bisulfite, TET2, T4-BGT, APOBEC, etc.) that causes a portion of the cytosines in the cfDNA to be converted to uracils. In some cases (e.g., traditional methylation sequencing (bisulfite sequencing as an example or enzymatic-methyl sequencing (EM-seq) as another example), the portion converted to uracils includes unmethylated cytosines. In some examples (e.g. TET-assisted pyridine borane sequencing (TAPS), the portion converted to uracils includes methylated cytosines. The remaining portion of the cytosines in the cfDNA remain as cytosines, for instance.

When the converted cfDNA is amplified, the amplified cfDNA includes thiamines at the positions of the converted uracils and includes cytosines at the positions of the unconverted cytosines. When the converted cfDNA is subsequently sequenced by the sequencer 112 to generate the sequence read data 114, the sequencer 112, for instance, may compare the sequences of the cfDNA indicated in the sequence read data 114 to at least one reference sequence (e.g., a reference genome) in order to identify which of the cytosines have been converted (e.g., to uracil). In various implementations, the sequencer 112 may identify which of the cytosines in the cfDNA in the liquid biopsy sample 106 were methylated based on the comparison of the cfDNA sequences in the sequence read data 114 to the reference sequence(s). In various implementations, indications of the position, order, and amount of the methylated cytosines is further indicated in the sequence read data 114. This information may be referred to as “methylation data.”

In various implementations, sequences representing the ctDNA 108 and sequences representing the non-ctDNA 110 in the sequence read data 114 are differentiated from one another. For instance, the sequences representing the non-ctDNA 110 may be removed from the sequence read data 114. In some examples, the sequencer 112 and/or another computing device removes the sequences representing the non-ctDNA 110 from the sequence read data 114. For ease of explanation, FIG. 1 will be described such that the sequencer 112 identifies the sequences belonging to the ctDNA 108, but implementations are not so limited.

Various features can be used to identify sequences corresponding to the ctDNA 108 rather than the non-ctDNA. In various implementations, the sequencer 112 identifies the sequences corresponding to the ctDNA 108 based on the lengths of the sequences indicated by the sequence read data 114. For instance, sequences with lengths over a predetermined threshold may be defined as corresponding to the ctDNA 108. In various examples, the sequencer 112 identifies sequences corresponding to the ctDNA 108 based on the presence of one or more predetermined variants associated with cancer. In various implementations, the sequencer analyzes the sequences of the fragments represented by the sequence read data 114 in order to determine which of the sequences correspond to the ctDNA 108.

A methylation analyzer 116 determines a methylation status 118 (also referred to as a “methylation state”) of the ctDNA 108 by analyzing the sequence read data 114. In various cases, the methylation analyzer 116 determines the methylation status 118 based on the sequences and methylation data of the cfDNA and/or the ctDNA 108 indicated in the sequence read data 114.

In various implementations, the methylation status 118 includes at least one metric indicating an amount of methylated cytosines in one or more regions of the ctDNA 108. In some examples, the implementation may be limited to CpG contexts (i.e. a cytosine followed by a guanosine) since these may be the most common locations to see methylated cytosines in human DNA. In other examples, the implementation may include all cytosines in a region, as some cancer aberrations involve methylating non-CpG cytosines. For instance, the methylation status 118 indicates a percentage or fraction of methylated cytosines with respect to the total number of cytosines in CpG contexts in the region(s) of the ctDNA 108. In some examples, the methylation status 118 indicates a percentage or fraction of methylated cytosines with respect to the total number of nucleotides within the region(s) of the ctDNA 108, which may also be referred to as a “density” of the methylated cytosines in the region(s). According to various cases, the methylation status 118 includes a total number of methylated cytosines in the region(s). In some cases, the methylation status 118 includes a running average of the density of methylated cytosines along the region(s) of the ctDNA 108 (e.g., a density of methylated cytosines within 5 nucleotides of a position, along each position of the region(s)). Other metrics characterizing the amount of methylated cytosines in the region(s) of the ctDNA 108 are also possible, and not limited to the specific examples described here.

In some cases, the methylation status 118 is representative of an amount of methylated cytosines observed in the cfDNA or ctDNA 108 over various genomic positions. For example, the methylation status 118 may be representative as a graph, histogram, or waveform plotted across the nucleotides in the region(s) of the ctDNA 108, such that square-shaped regions of the methylation status may be representative of highly methylated portions of the region(s).

According to some cases, the methylation analyzer 116 determines the methylation status of the region(s) in the cfDNA (including a methylation status of the ctDNA 108 and the non-ctDNA 110) and generates the methylation status 118 of the region(s) in the ctDNA 108 based on the methylation status of the cfDNA and a tumor-fraction-dependent correction. For instance, the methylation status of the region(s) in the genome of at least one individual (not the subject 102) without cancer may be known. In addition, the methylation analyzer 116 may determine a tumor fraction of the cfDNA. Based on the methylation status of the region(s) in the genome of the individual(s) without cancer, the tumor fraction, and the methylation status of the region(s) in the cfDNA, the methylation analyzer 116 may determine the methylation status 118 of the region(s) in the ctDNA 108.

In various implementations, the methylation analyzer 116 identifies a tumor fraction of the cfDNA. The tumor fraction, for instance, represents the portion of the cfDNA that includes the ctDNA 108. Various techniques can be performed in order to calculate tumor fraction. In various examples, tumor fraction is a measure of an amount of the ctDNA 108 relative to the amount of cfDNA in the liquid biopsy sample 106. Tumor fraction can be determined using a variety of techniques, such as by inferring purity and ploidy from log ratio and/or allele frequency measurements. The log ratio and/or allele frequency measurements, for instance, may be determined by analyzing the sequence read data 114.

In some embodiments, tumor cell ploidy can be used to calculate tumor fraction. Tumor cell ploidy, for instance, can refer to the average weighted copy number of all chromosomes (or portions thereof) in the sequence read data 114. In some cases, the tumor fraction is determined based on the allele coverage or allele fraction at one or more subgenomic intervals in the cfDNA. The subgenomic interval(s) for instance, include one or more heterogenous single nucleotide polymorphisms (SNPs) and/or intervals that are longer than a single nucleotide. The term “allele fraction,” as used herein, refers to the relative level (e.g., abundance) of an allele at a subgenomic interval in a sample. In particular examples, the sequence read data 114 may indicate multiple SNPs indicating cfDNA fragments with different nucleotide types at particular positions relative to a reference genome. An allele fraction for each SNP may be determined (e.g., a fraction of the cfDNA fragments with C at a given genomic position, a fraction of the cfDNA fragments with T at the given position, etc.). In various cases, a computing model can be utilized to determine tumor fraction of the liquid biopsy sample 106 based on the allele fractions of the SNPs (or longer subgenomic intervals) within the cfDNA of the liquid biopsy sample 106.

In particular cases, the methylation analyzer 116 determines the methylation status 118 of the region(s) in the ctDNA 108 using the following Equation 1:

$${m}_{t} = \frac{{m}_{p} - {m}_{0}}{f} + {m}_{0} \qquad ( 1 )$$

wherein mₜ is the methylation status 118 as a fraction of the subject's 102 ctDNA 108 in the region(s), mₚ is a methylation status of the region(s) in the cfDNA, f is the tumor fraction of the liquid biopsy sample 106, and m₀ includes a methylation status of the region(s) in a genome of the individual(s) without cancer. Therefore, in some cases, the methylation status 118 of the ctDNA 108 may be derived without isolating sequences of the ctDNA 108 and the non-ctDNA 110 in the sequence read data 114.

In various cases, the region(s) in the ctDNA 108 are indicative of expression by cancer cells in the lesion 104. Examples of region(s) of interest include at least a portion of a gene-of-interest, at least a portion of a promoter operably linked with the gene, at least a portion of an enhancer operably linked with the gene, or any combination thereof. In some cases, the region(s) of interest include at least a portion of a CpG island. For instance, the promoter, the enhancer, the CpG island, or any combination thereof, is within a threshold distance (e.g., 100 bases) of the gene.

The methylation status 118 of the region(s) in the ctDNA 108, in various cases, are indicative of the expression of one or more sequences by the cancer cells in the lesion 104. For example, the sequence(s) include the gene. In various cases, the expression of the sequence(s) is related to one or more expression pathways of the cancer cells in the lesion 104.

To determine the expression of the sequence(s), a predictive model 120 is configured to generate one or more expression indicators 122 based on the methylation status 118. In some cases, the predictive model 120 further analyzes additional biomarker data in order to generate the expression indicator(s) 122. For instance, the predictive model 120 may receive input data including the methylation status 118 as well as data indicating at least one of a genomic alteration, a mutational signature, an MSI status, a TMB, or a viral status of the subject 102 and/or lesion 104. The additional biomarker data may be generated based on the liquid biopsy sample 106, medical images, or other samples obtained from the subject 102.

The predictive model 120, for example, may include one or more mathematical and/or computer-based models that are configured to predict the expression of the sequence(s) by the cancer cells based on the methylation status 118. For instance, the predictive model 120 may include a regression model, threshold rule, confidence interval, or other type of statistical model capable of categorizing the cancer based on the methylation status 118.

In various implementations, the predictive model 120 includes at least one trained ML model configured to output the expression indicators 122 in response to receiving the methylation status 118 in input data. For example, parameters of the ML model(s) may have been previously optimized based on training data including the methylation status of regions in genomes of individuals within a population omitting the subject 102. For instance, the ML model(s) was trained using an unsupervised or semi-supervised learning technique, wherein the parameters were optimized to categorize (e.g., cluster) the methylation statuses of the population. In some cases, the ML model(s) was trained using a supervised learning technique, wherein the training data further included ground truth categorizations of the expression of the sequence(s) of cancer cells of the individuals in the population, such that the parameters were optimized to minimize a loss between predicted expression indicators generated by the ML model(s) based on the methylation statuses of the population and the ground truth expression indicators of the individuals in the population. To increase training robustness, the population represented by the training data may include individuals without cancer, as well as individuals with a variety of cancer types and metastasis states. Various types of ML models can be included in the predictive model 120, such as a neural network (e.g., a convolutional neural network (CNN)), a nearest-neighbor model, a regression analysis model, a clustering model, a principal component analysis model, a gradient boosting model, a random forest, or any combination thereof.

The expression indicators 122 may indicate a probability that the cancer cells of the subject 102 express the sequence(s). For example, the predictive model 120 may determine a likelihood that the cancer cells of the subject 102 participate in a given expression pathway. In various cases, the methylation status of the region(s) is indicative of whether the cancer cells express the sequence(s). For instance, a highly methylated promoter (or enhancer) may prevent the expression of a gene that is operably coupled to the promoter (or enhancer). In some cases, highly methylated portions of a gene may enhance expression of the gene.

The expression indicator(s) 122 may, in some cases, indicate whether the cancer of the subject 102 is resistant or responsive to one or more predetermined therapies. In various cases, the expression of the sequence(s) by the cancer cells indicated in the ctDNA 108 is indicative of whether the cancer cells are resistant (e.g., at least partially unharmed) if a particular therapy is administered, or whether the cancer cells are responsive (e.g., at least partially killed or otherwise destroyed) if a particular therapy is administered. In various implementations, the predictive model 120 determines whether each of one or more therapies is likely to successfully treat the cancer of the subject 102.

According to some cases, the predictive model 120 is configured to determine whether the subject 102 qualifies for a study, such as a clinical trial. For example, the predictive model 120 may determine that the subject 102 has cancer cells that express the sequence(s) and may therefore enroll in a clinical trial to investigate the efficacy of a new therapy (e.g., a new immunotherapy). The expression indicator(s) 122, for instance, indicate whether the subject 102 qualifies for the clinical trial.

In some implementations, the predictive model 120 is unable to conclusively determine that the cancer cells express the sequence(s) of interest. For example, the predictive model 120 may determine that, based on the methylation status 118, the certainty of the probability that the cancer cells express the sequence(s) is below a threshold certainty. In various cases, the expression indicator(s) 122 may indicate that the expression of the sequence(s) is inconclusive.

A report generator 124 is configured to generate a report 126 based on the category indicator(s) 122. The report 126, for example, includes consumable data that can inform the care provider 105 about the at least one determined category of the cancer of the subject 102. Further, in some cases, the report 126 indicates whether the lesion 104 of the subject 102 is cancerous by reporting whether the ctDNA 108 has been identified in the liquid biopsy sample 106. In various implementations, the report 126 may indicate the results of additional analyses, such as the results of a histological study, whole transcriptome sequencing, cfRNA sequencing, whole exome sequencing, whole genome sequencing, a cancer (e.g., DNA) hotspot panel test, a DNA methylation test, a tumor mutational burden (TMB) test, a DNA fragmentation test, an RNA fragmentation test, a microsatellite instability (MSI) test, a tumor mutational burden (TMB) test, or a viral status test. The performance of such tests is within the ordinary skill of the art, with additional detail provided elsewhere herein. The report 126, for example, may include a genomic profile of the subject 102 based on various combinations of the above analyses and tests.

In some implementations, the report 126 indicates that a follow-up test of the subject 102 is indicated. For instance, in response to determining that the categorization of the cancer is inconclusive, the report generator 124 may generate the report 126 to indicate that one or more additional tests (e.g., a histological study, genome sequencing, exome sequencing, additional DNA sequencing, RNA sequencing, transcriptome sequencing, etc.) should be performed in order to identify whether the cancer cells of the subject 102 express the sequence(s).

In various cases, the report 126 is output to a clinical device 128. For example, the report generator 124 transmits the report 126 to the clinical device 128. In various implementations, the clinical device 128 is a computing device that is operated by, owned by, or otherwise associated with the care provider 105. For instance, the clinical device 128 may be a desktop computer, a laptop computer, a smart phone, or some other computing device associated with the care provider 105. The clinical device 128, in various cases, outputs the report 126 to the care provider 105. In some cases, the clinical device 128 includes a display (e.g., a screen) that visually presents the report 126. In various cases, the clinical device 128 includes a speaker that outputs a sound indicative of the report 126. The clinical device 128, in various cases, may output the information in the report 126 using one or more output mechanisms or devices.

The care provider 105 may review the report 126 by interacting with the clinical device 128. The report 126, in various cases, may enhance the clinical decision-making of the care provider 105. For instance, the care provider 105 may prepare and/or administer a therapy to the subject 102 based on the report 126. According to various implementations, the care provider 105 may initiate the therapy and/or refer the subject 102 to another care provider to receive the therapy.

In various implementations, the care provider 105 may develop a diagnosis and/or prognosis of the subject 102 based on the report 126. In various implementations, the care provider 105 may communicate information in the report 126 to the subject 102.

FIG. 1 illustrates various elements that can be embodied in one or more computing devices. For example, at least a portion of the functions of the sequencer 112, the methylation analyzer 116, the predictive model 120, the report generator 124, and the clinical device 128 are performed by one or more processors in at least one computing device. Examples of computing devices include server computers, desktop computers, laptop computers, tablet computers, mobile phones, wearable devices, Internet of Things (IoT) devices, and the like. In various cases, instructions for performing at least a portion of the functions of these elements are stored in memory and/or in a non-transitory computer readable medium. The instructions, for instance, are executed by the processor(s).

FIG. 1 also illustrates various types of data. For example, the sequence read data 114, the methylation status 118, the expression indicator(s) 122, the report 126, or any combination thereof, includes data. The various types of data illustrated in FIG. 1 may be stored, such as in memory or in non-transitory computer readable media. In various implementations, at least a portion of the data is transmitted or otherwise output by one or more computing devices. For example, a computing device may transmit one or more communication signals to another computing device, wherein the communication signal(s) encode at least a portion of the data. Examples of communication signals include electromagnetic signals, optical signals, ultrasonic signals, optical signals, and electrical signals. For example, communication signals can be transmitted wirelessly and/or in a wired fashion. The communication signals, for instance, are transmitted over one or more wireless channels and/or one or more wired channels (e.g., optical cabling, electrical cabling, etc.). In various cases, the communication signal(s) are transmitted over one or more communication networks. A communication network, for instance, may be defined according to one or more physical channels, such as one or more frequency spectra. In some cases, a communication network is defined according to one or more communication protocols and/or standards. Examples of communication networks include fiber optic networks, Institute of Electrical and Electronics Engineers (IEEE) networks (e.g., WI-FI™ networks, WiMAX networks, BLUETOOTH™ networks, etc.), cellular networks (e.g., a 3^{rd} Generation Partnership Project (3GPP) radio network, such as a Long Term Evolution (LTE) network, a New Radio (NR) network; or a cellular core network such as a 3^{rd} Generation (3G) core, a 4th Generation (4G) core, a 5th Generation (5G) core, etc.), ultrasonic networks, and the like. In some cases, the data is broadcasted from one device to multiple other devices. In some cases, the data is unicasted from one device to another device. For instance, various forms of data described herein may be transmitted via a peer-to-peer (P2P) connection.

FIG. 2 illustrates an example environment 200 illustrating ctDNA 202, which can be utilized to analyze cancer cells of a subject. For instance, the ctDNA 202 may be the ctDNA 108 described above with reference to FIG. 1.

In various implementations, a cancer cell 204 within the subject includes genomic DNA (gDNA) that is expressed by the cancer cell 204. For example, the gDNA 206 may include various sequences, such as a gene 208, a promoter 210, an enhancer 212, and a variant 214. For example, the variant 214 is part of the gene 208. In addition, various epigenetic factors impact expression of the gene 208 as well as other genes within the gDNA 206. For example, the gDNA 206 may be packaged within the nucleus of the cancer cell 204 with various histones 216. When the gene 208 is expressed, a portion of the gDNA 206 including the gene 208, the promotor 210, the enhancer 212, and the variant 214 may be exposed to proteins within the nucleus, such as RNA transcriptase. In various cases, the portion of the gDNA 206 is unwrapped or otherwise unpackaged from the histones 216. Thus, the expression of the gene 208 (e.g., the amount of mRNA generated by RNA transcriptase based on the gene 208 within the cancer cell 204) is linked to the frequency or time at which the portion of the gDNA 206 is exposed.

The cancer cell 204, for example, may die. The contents of the cancer cell 204, including the gDNA 206, may be released. In various cases, the gDNA 206 is released into blood 218 that flows through a blood vessel 220 of the subject. When the gDNA 206 is released from the nucleus of the cancer cell 204, the gDNA 206 is degraded due to various biophysical and/or biochemical factors. For example, the blood 218 may include various enzymes that cut the gDNA 206 into the ctDNA 202. In various cases, other mechanical, chemical, or thermal conditions in the blood 218 divide the gDNA 206 into the ctDNA 202. For example, these conditions divide the gDNA 206 into fragments at various breakpoints 222.

Notably, the presence and location of the histones 216 may impact the sequences of the ctDNA 202 that are observed in the blood 218. The breakpoints 222, for example, are more likely to occur at edges of a sequence of the gDNA 206 that is exposed by the histones 216. Therefore, the sequence of the ctDNA 202, in various examples, is indicative of the expression of mRNA and other functional RNA in the cancer cell 204. By reviewing the ctDNA 202, the expression of the cancer cell 204 can be determined without performing RNA sequencing, in some cases.

However, the methylation status of various regions within the ctDNA 202 are also indicative of the expression of the cancer cell 204. For example, even though the promoter 210 is operatively coupled to the gene 208 (e.g., the promoter 210 includes a transcription start site of the gene 208), and the promoter 210 is present in the ctDNA 202, certain epigenetic factors of the promoter 210 may indicate that the gene 208 is not expressed by the cancer cell 204. For instance, the promoter 210 may include various cytosines that are methylated. The methylated cytosines, in various cases, may have prevented RNA polymerase from binding to the promoter 210 when the promoter 210 was part of the gDNA 206, thereby preventing expression of the gene 208 in the cancer cell 204. In these cases, an anticancer therapy that targets cells expressing the gene 208 may be ineffective at targeting the cancer cell 204. Thus, in various implementations of the present disclosure, the methylation status of various regions (including, e.g., the gene 208, the promoter 210, the enhancer 212, the variant 214, etc.) in the ctDNA 202 may be determined.

In various implementations, the ctDNA 202 is obtained from a sample of plasma 232 in the blood 218 of the subject. The plasma 232, for example, includes various DNA fragments 234 including the ctDNA 202. In some cases, the DNA fragments 234 include various cfDNA, such as cfDNA released from non-cancerous cells.

By sequencing the ctDNA 202 using methylation sequencing, the methylation status of one or more region(s) can be determined. The methylation status can be utilized to determine whether the cancer cell 204 expresses one or more sequences-of-interest (e.g., genes). Thus, it may be determined whether the cancer cell 204 is responsive to a therapy targeting cells that express the sequence(s)-of-interest.

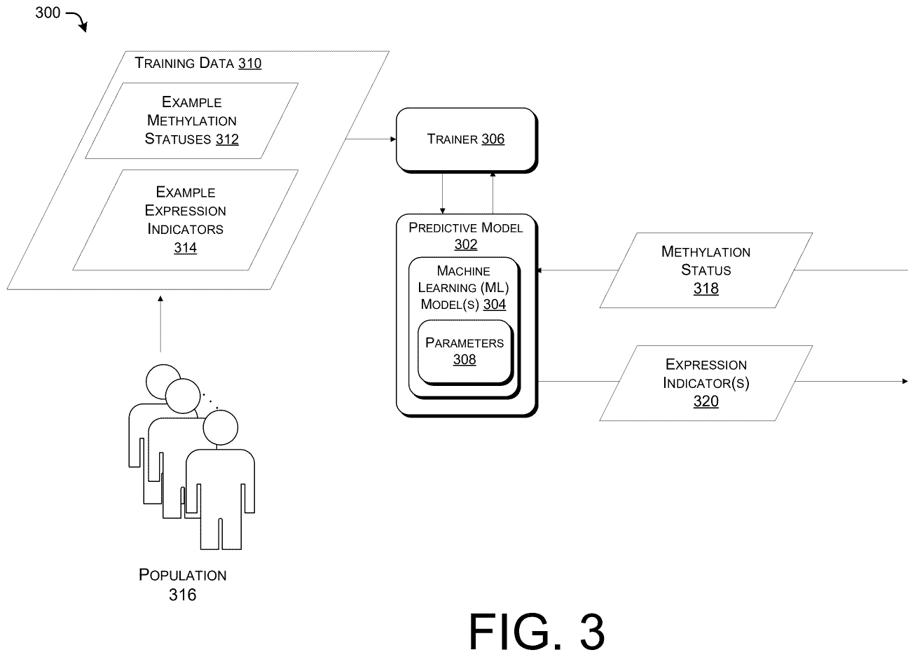

FIG. 3 illustrates an example environment 300 for training and utilizing a predictive model 302 to determine expression of cancer cells based on methylation statuses of regions of DNA derived from the cancer cells. The predictive model 302, for instance, is the predictive model 120 described above with reference to FIG. 1. In various implementations, the predictive model 302 includes one or more ML models 304. A trainer 306, for instance, is configured to optimize various parameters 308 of the ML model(s) 304 based on training data 310.

The training data 310 includes example methylation statuses 312 and example expression indicators 314. The example methylation statuses 312, in various cases, are obtained based on ctDNA of individuals within a population 316. For instance, the methylation statuses 312 are indicative of one or more regions of interest within the ctDNA. The example expression indicators 314 may indicate whether cancer cells of the individuals within the population 316 express one or more sequences. For example, the example expression indicators 314 may be generated based on samples obtained from the individual that are not limited to ctDNA. In some cases, the example expression indicators 314 are obtained by performing whole genome sequencing, whole exome sequencing, RNA sequencing, immunohistochemical studies, post-immunotherapy treatment analyses, or other types of analyses. In various cases, the population 316 includes individuals with different types of cancers, different types of severities, and the like.

The ML model(s) 304 include one or more model types. For instance, the ML model(s) 304 include an artificial neural network. An artificial neural network includes various layers that respectively process input data. For example, an artificial neural network includes an input layer, one or more hidden layers, and an output layer. The input layer performs a pre-processing operation on the input data. The hidden layer(s) may perform various processing operations on the output from the input layer. The output layer, in various cases, processes the output from the hidden layer(s). Each layer, in some cases, includes one or more nodes, which are defined by individual operations. In various cases, the hidden layer(s) include nodes that are connected to each other in parallel and/or series. Examples of artificial neural networks include feedforward neural networks, multi-layer perceptrons (MLPs), convolutional neural networks (CNNs), and backpropagation models. In various implementations, the operations performed by the layers and/or nodes within an artificial neural network included in the ML model(s) 304 is defined according to the parameters 308. For example, the parameters 308 may include weights, thresholds, filters, kernels, or other data objects that are utilized to perform operations of the ML model(s) 304.

In some implementations, the ML model(s) 304 include a nearest-neighbor model. One example of a nearest-neighbor model includes a k-nearest neighbor model. For example, a nearest-neighbor model defines various “neighbors,” which are points within a feature space, with associated class labels. When a new data point is mapped to the feature space, the new data point is classified based on the proximity (e.g., Euclidian distance, Manhattan distance, Minkowski distance, etc.) of its “neighbors” to the new data point as well as their associated classes. In some cases, the new data point is classified as belonging to a particular class if greater than a threshold number of neighbors within a threshold distance of the new data point are members of the class. For instance, the parameters 308 may include k (e.g., the number of neighbors compared to the new data point), the threshold distance, and so on.

In various cases, the ML model(s) 304 include a regression analysis model. The regression analysis model, for example, is defined by a regression function that defines relationships between one or more independent variables and one or more dependent variables. The regression function may further define one or more unknown parameters that define a relationship between the independent and dependent variables. In various implementations, the unknown parameters and/or the type of regression function (e.g., linear, quadratic, etc.), is defined according to the parameters 308.

In some cases, the ML model(s) 304 include a clustering model. In various cases, a clustering model maps various data points (e.g., training data) to a feature space. Based on the proximity of groups of those data points in the features pace, one or more “clusters” are defined. An additional data point may be classified according to one or more of the clusters based on its proximity to the clusters (e.g., a center of the clusters, a boundary of the cluster, etc.). Examples of clustering models include k-means clustering, mean-shift clustering, expectation-maximization (EM) clustering, and agglomerative hierarchical clustering. The parameter(s) 308, for example, include a threshold proximity within which a new data point is classified within a cluster, a density of points used to define a cluster, and the like.

In various examples, the ML model(s) 304 include a principal component analysis model. In various implementations, a principal component analysis defines a collection principal components of unit vectors within a coordinate space based on a data set (e.g., training data). The model, for example, is an orthogonal linear transformation of the data set. Various weights of the model, for example, are included in the parameter(s) 308.

The ML model(s) 304, in some implementations, includes a gradient boosting model. For example, the gradient boosting model is defined as a collection of prediction models (e.g., decision trees) that iteratively classify observed data. In various cases, the type of prediction model, weights in the prediction models, and the like, are defined by the parameter(s) 308.

The ML model(s) 304, for example, includes a random forest. The random forest, for instance, includes multiple decision trees that classify data in an ensemble fashion. In various implementations, the decision trees are defined by the parameter(s) 308.

In various implementations of the present disclosure, the trainer 306 is configured to optimize the parameters 308 based on the training data 310. For example, the trainer 306 may input first example methylation status (corresponding to a first individual among the population 316) among the example methylation statuses 312 into the predictive model 302, and may receive a predicted category. The trainer 306 may compute a loss (e.g., determine a discrepancy) between a first example expression indicator (corresponding to the first individual) among the example expression indicators 314 and the predicted category. Further, the trainer 306 may alter the parameters 308 in order to minimize the loss. In various cases, the trainer 306 optimizes the parameters 308 iteratively based on the entire set of the training data 310.

In various implementations, the optimization of the parameters 308 enables the predictive model 302 to identify predictive attributes of the example methylation statuses 312 that are correlated to or otherwise associated with the example expression indicators 314. For instance, the predictive model 302 may determine that a methylation fraction above 80% in a particular promoter represented in the example methylation statuses 312 is highly correlated with limited expression of KRAS. The predictive model 302 may therefore determine whether a methylation status outside of the example methylation statuses 312 is indicative of expression of KRAS by recognizing or otherwise identifying the predictive attributes.

Once the parameters 308 are optimized, the predictive model 302 may be ready to classify a new set of data. For example, the predictive model 302 may receive input data including a methylation status 318 of a subject. The methylation status 318, for instance, may include one or more of the predictive attributes. The predictive model 302 may perform various operations on the input data based on the trained ML model(s) 304 and the optimized parameters 308. In various cases, the predictive model 302 outputs output data including one or more expression indicators 320 based on the methylation status 318. The expression indicator(s) 320, for instance, indicate whether a particular therapy is predicted to be effective in treating the cancer cells of the subject.

Although FIG. 3 is primarily described as referring to supervised learning, implementations are not so limited. In various cases, the training data 310 omits the example expression indicators 314 and the trainer 306 is configured to optimize the parameters 308 using the example methylation statuses 312 and an unsupervised learning technique.

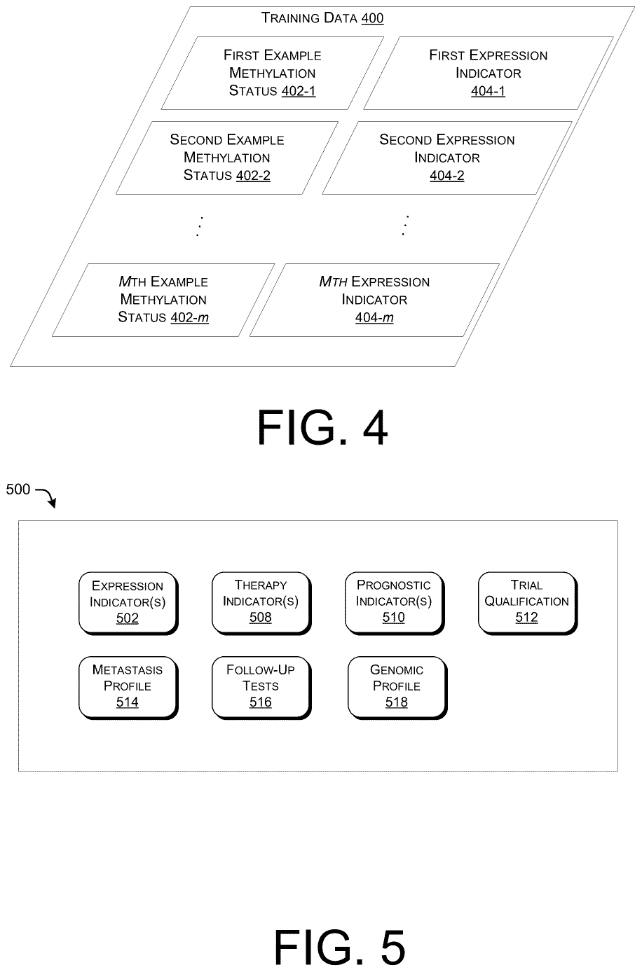

FIG. 4 illustrates an example of training data 400 utilized to train one or more ML models. For example, the training data 400 may be the training data 310 described above with reference to FIG. 3.

The training data 400, in various cases, may represent m samples, wherein m is a positive integer. In some cases, the m samples are respectively obtained from m individuals within a population, although implementations are not so limited. For example, in some cases, multiple samples may be obtained from the same individual at different times.

The training data 400 includes first to mth example methylation statuses 402-1 to 402-m. For example, the first to mth example methylation statuses 402-1 to 402-m include methylation statuses of one or more regions in cfDNA (e.g., ctDNA) obtained from the respective m samples.

The training data 400 may further include first to mth example expression indicators 404-1 to 404-m. The first to mth example expression indicators 404-1 to 404-m, for instance, include indications of whether cancer cells represented by the m samples express one or more predetermined sequences (e.g., genes).

FIG. 5 illustrates an example report 500 summarizing predicted categories of a cancer of a subject. In various cases, the report 500 is the report 126 described above with reference to FIG. 1. The report 500, for instance, may be displayed to a patient and/or care provider. In some cases, the report 500 is generated based on a methylation status of one or more regions in a sample (e.g., a liquid biopsy sample) obtained from the subject.

The report 500 includes an expression indicator 502 of the cancer. The expression indicator 502 indicates whether cancer cells of the subject express one or more sequences-of-interest.

In various cases, the report 500 includes one or more therapy indicators 508. For instance, the therapy indicator(s) 508 convey whether the cancer is predicted to be resistant to one or more predetermined therapies and/or whether the cancer is predicted to be responsive to one or more predetermined therapies.

In some examples, the report 500 includes one or more prognostic indicators 510. The prognostic indicator(s) 510, for instance, indicate a prognosis of the subject. For example, the prognostic indicator(s) 510 may indicate a survivability, a recoverability, a quality of life indicator, or other information indicative of the prognosis of the subject.

The report 500 may include a trial qualification 512 of the subject. The trial qualification 512, for instance, indicates whether the subject is predicted to qualify for a predetermined clinical trial.

The report 500, in various implementations, includes a metastasis profile 514 of the subject. The metastasis profile 514, for instance, indicates a likelihood that the cancer will metastasize (e.g., at a particular point in time), one or more tissues in which the cancer is predicted to metastasize, or the like.

In various cases, the report 500 includes recommended follow-up tests 516. For example, the report 500 may include a recommendation to perform whole genome sequencing on the subject, particularly in cases if the cancer cannot be categorized above a threshold certainty.

The report 500 may include a genomic profile 518 of the subject. In various cases, the genomic profile 518 includes or is generated based on the results of non-methylation analyses of the subject.

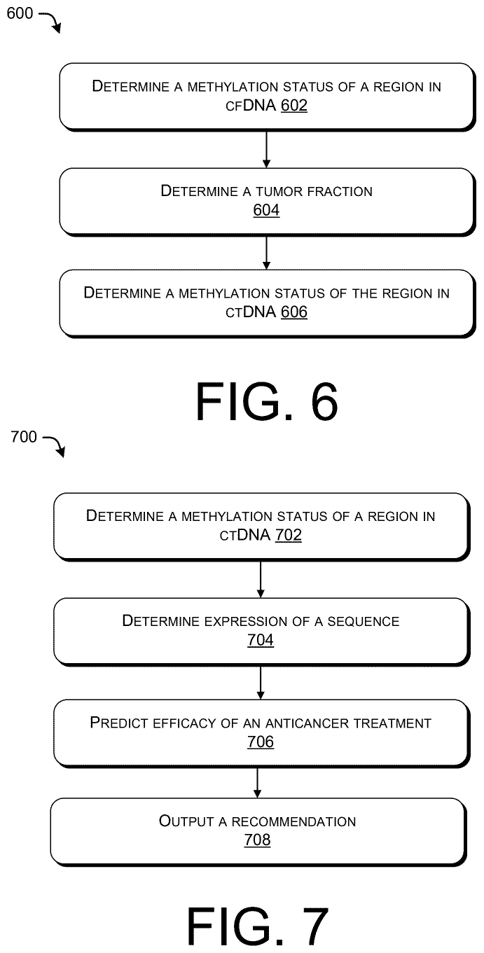

FIG. 6 illustrates an example process 600 for determining a methylation status of a sample. The process 600, in various examples, is performed by an entity, such as at least one computing device, at least one processor, the sequencer 112, the methylation analyzer 116, the predictive model 120, the report generator 124, the clinical device 128, or any combination thereof.

At 602, the entity determines a methylation status of a region in cfDNA of a sample. In various implementations, the entity extracts the cfDNA from the sample. For instance, the sample is a liquid biopsy sample (e.g., a serum sample). In various cases, the entity sequences the cfDNA. For example, the entity may perform methylation sequencing on the cfDNA. In some cases, the entity converts methylated (or unmethylated) cytosines in the cfDNA into uracils, and then sequences the converted cfDNA to obtain sequence reads of the cfDNA. During an amplification process, the uracils may be copied as thiamines. By comparing the sequences of the cfDNA to one or more reference sequences (e.g., one or more reference genomes), the entity can infer which thiamines indicated in the sequences of the converted cfDNA are indicative of converted cytosines. Accordingly, the entity may determine which of the cytosines in the cfDNA obtained from the sample were methylated. In various implementations, the entity may determine the methylation status based on an amount, percentage, density, presence, or distribution of methylated cytosines in the region of the cfDNA. The region, for example, may be at least a portion of a promoter operably coupled to a gene, at least a portion of an enhancer operably coupled to the gene, at least a portion of the gene, at least a portion of a CpG island associated with the gene, or any combination thereof.

At 604, the entity determines a tumor fraction of the sample. In various implementations, the entity determines the tumor fraction by determining how much of the cfDNA is ctDNA and/or how much of the cfDNA is non-ctDNA. In some cases, the entity determines the tumor fraction based on an abundance of alleles at various subgenomic intervals of the cfDNA. For example, the entity may determine a certainty metric based on the allele fraction at each of multiple subgenomic intervals in the cfDNA. Based on a predetermined (e.g., stored) relationship between the certainty metric and the allele fraction, the entity may determine the tumor fraction of the sample. For example, the predetermined relationship may be stored in a trained ML model.

At 606, the entity determines a methylation status of the region in ctDNA of the sample based on the methylation status of the region in the cfDNA and the tumor fraction. In various cases, the entity may determine a correction of the methylation status based on the tumor fraction. In some examples, the correction is further based on a known methylation status of one or more individuals without cancer. For instance, the entity may apply Equation 1 in order to identify the methylation status of the region in the ctDNA of the sample.

FIG. 7 illustrates an example process 700 for recommending an anticancer treatment based on a methylation status of a sample. The process 700, in various examples, is performed by an entity, such as at least one computing device, at least one processor, the sequencer 112, the methylation analyzer 116, the predictive model 120, the report generator 124, the clinical device 128, the care provider, or any combination thereof.

At 702, the entity determines a methylation status of a region in ctDNA of a sample of a subject. For instance, the entity may receive the methylation status from an external device. In some cases, the entity calculates the methylation status, such as by performing the process 600 described above with reference to FIG. 6.

At 704, the entity determines an expression of a sequence based on the methylation status of the region in the ctDNA. In various implementations, the region is at least a portion of the sequence and/or is operably coupled to one or more genes associated with the sequence. For instance, the region is within a threshold distance (e.g., within 1, 5, 10, 50, 100, or 200) nucleotides of the sequence-of-interest. In various cases, the methylation status is included in input data. A model (e.g., including one or more ML models) may be configured to output an indicator of the expression based on receiving the input data. For instance, the model may output a probability that cells within a tumor associated with the ctDNA express the sequence.

At 706, the entity predicts that the anticancer treatment would be effective based on the expression of the sequence. In various implementations, the anticancer treatment targets cells that express the sequence-of-interest. In particular examples, the methylation status of the region indicates that the sequence is expressed. For example, a promoter operably coupled to the sequence has less than a threshold methylation fraction. Based on the indicator of the expression of the sequence, the entity may predict that the anticancer treatment would be effective.

At 708, the entity outputs a recommendation to administer the anticancer treatment to the subject. In various cases, the entity outputs the recommendation in a report associated with the subject. A care provider, for instance, may prepare and/or administer the anticancer treatment to the subject based on the report.

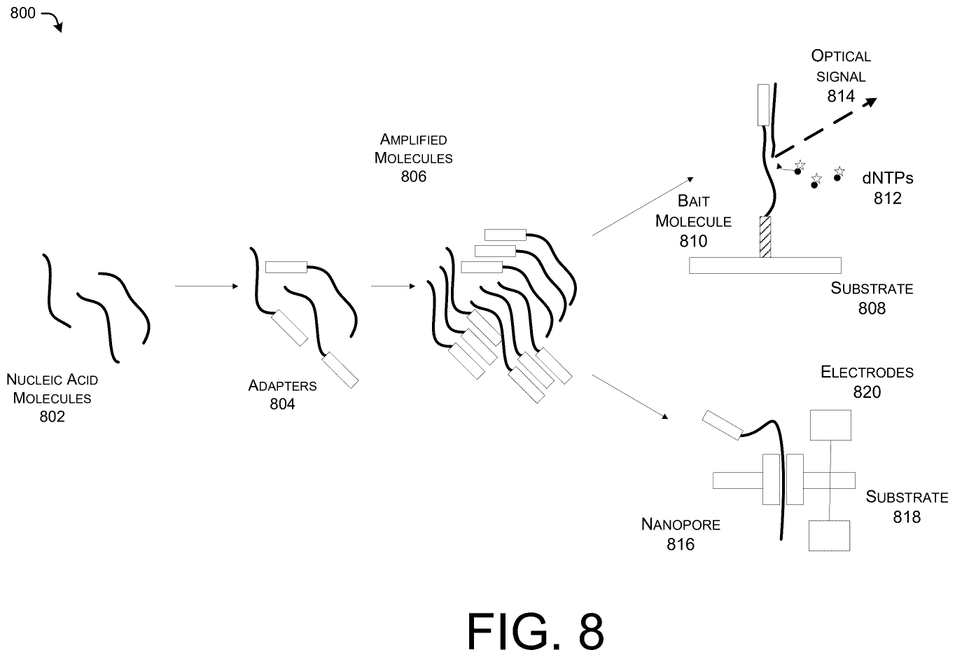

FIG. 8 illustrates an example environment 800 for sequencing various nucleic acid molecules 802. In various implementations, the nucleic acid molecules 802 include cfDNA and/or gDNA. For instance, the nucleic acid molecules 802 may include ctDNA. The nucleic acid molecules 802, in various cases, are extracted from a sample, such as a biological sample obtained from a subject. In some implementations, the nucleic acid molecules 802 include DNA that is complementary to RNA present in the sample.

In various implementations, the nucleic acid molecules 802 are subjected with a treatment that causes conversion of at least some of the cytosines in the nucleic acid molecules 802 to be converted to uracil. In some implementations, bisulfite is used to convert unmethylated cytosines in the nucleic acid molecules 802 into uracils.

Alternatively, in some cases, methylated cytosines in the nucleic acid molecules 802 are converted into uracils using at least a two-step process. Initially, at least one first enzyme (e.g., tet methylcytosine dioxygenase 2 (TET2) and/or T4-phage beta-glucosyltransferase (T4-BGT)) converts methylated cytosines in the nucleic acid molecules 802 into a protected form (e.g., 5-(β-glucosyloxymethyl) cytosine (5gmC). Next, at least one second enzyme (e.g., apolipoprotein B mRNA editing enzyme catalytic subunit 3A (APOBEC3A) is used to convert the unmethylated and unmodified cytosines into uracils.

The nucleic acid molecules 802, in various cases, are ligated with adapters 804. For examples, the adapters 804 are hybridized to the nucleic acid molecules 802. The adapters 804, for example, include additional nucleic acid molecules. In various implementations, the adapters 804 have a shorter length than the nucleic acid molecules 802 being sequenced. For instance, the adapters 804 include amplification primers, flow cell adapter sequences, substrate adapter sequences, or sample index sequences. Although FIG. 8 illustrates adapters 804 being ligated to one end of each of the nucleic acid molecules 802, implementations are not so limited. For example, the adapters 804 may be ligated to both ends of each of the nucleic acid molecules 802.

In various examples, the nucleic acid molecules 802 ligated with the adapters 804 are amplified in order to generate amplified molecules 806. Various amplification techniques can be performed. For instance, the amplified molecules 806 are generated using PCR, a non-PCR amplification technique, an isothermal amplification technique, or any combination thereof. In various implementations, during amplification, multiple copies of the nucleic acid molecules 802 are generated. However, the uracils in the treated nucleic acid molecules 802 may be copied as thiamines in the amplified molecules 806.

Amplified molecules 806 may be captured by bait molecules 810 and sequenced. In some implementations, the amplified molecules 806 are sequenced via sequencing-by-synthesis. In various cases, fluorescently tagged deoxyribonucleotide triphosphates (dNTP) 812 are utilized to synthesize a strand that is complementary to DNA strands bound to the substrate 808. When a dNTP 812 is added to the strand (e.g., by an enzyme), the dNTP 812 emits an optical signal 814. In various implementations, the frequency of the optical signal 814 is dependent on the type of dNTP 812 from which the optical signal 814 is emitted. By detecting the optical signals 814 as the strand is being synthesized, the sequence of the original nucleic acid molecules 802 can be derived.

In some implementations, the amplified molecules 806 are sequenced via nanopore sequencing. For instance, the amplified molecules 806 are directed through a nanopore 816 extending through a substrate 818. In various cases, the amplified molecules 806 are negatively charged, such that they can be directed through the nanopore 816 by imposing an electrical field across the substrate 818. In various cases, the amplified molecules 806 and the nanopore 816 are in the presence of a charged solution. Thus, charged solutes traveling through the nanopore 816 can be monitored by reviewing an electrical signal (e.g., a current) sensed between electrodes 820 on either side of the substrate 818. As an amplified molecule 806 is directed through the nanopore 816, the individual bases within the amplified molecule 806 will block the nanopore 816, which may decrease the amount of charged solutes traveling through the nanopore 816 and consequently, the magnitude of the electrical signal detected by the electrodes 820. Each of the four types of bases within the amplified molecules 806, may block the nanopore 816 to a different extent. Therefore, the sequence of the nucleic acid molecules 802 can be derived by analyzing the measured electrical signal with respect to time as the amplified molecules 806 are directed through the nanopore 816.

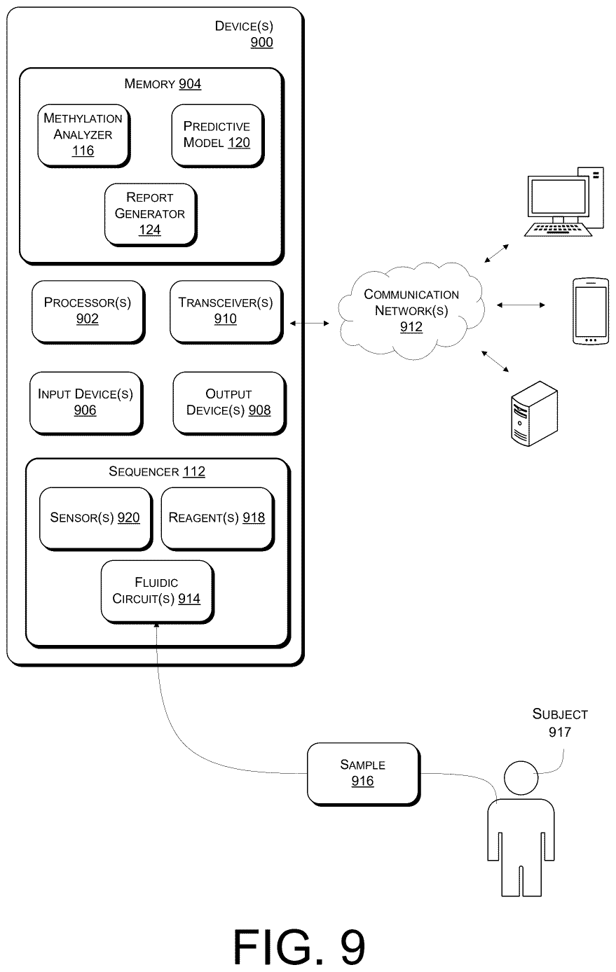

FIG. 9 illustrates one or more devices 900 configured to perform various operations described herein. The device(s) 900 include one or more processor(s) 902. In some implementations, the processor(s) 902 includes a central processing unit (CPU), a graphics processing unit (GPU), both CPU and GPU, or other processing unit or component known in the art.

The processor(s) 902 is operably connected to memory 904. In various implementations, the memory 904 is volatile (such as random access memory (RAM)), non-volatile (such as read only memory (ROM), flash memory, etc.) or some combination of the two. The memory 904 stores instructions that, when executed by the processor(s) 902, causes the processor(s) 902 to perform various operations. In various examples, the memory 904 stores methods, threads, processes, applications, objects, modules, any other sort of executable instruction, or a combination thereof. In some cases, the memory 904 stores files, databases, or a combination thereof. In some examples, the memory 904 includes, but is not limited to, RAM, ROM, electrically erasable programmable read-only memory (EEPROM), flash memory, or any other memory technology. In some examples, the memory 904 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 902. For instance, the memory 904 stores instructions that, when executed by the processor(s) 902, causes the processor(s) 902 to perform operations of the methylation analyzer 116, the predictive model 120, and the report generator 124.

The processor(s) 902 is operably connected to one or more input devices 906 and one or more output devices 908. Collectively, the input device(s) 906 and the output device(s) 908 function as an interface between at least one user and the device(s) 900. The input device(s) 906 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 908 includes at least one of a display, a speaker, a haptic output device, a printer, or any combination thereof. In various examples, the processor(s) 902 causes a display among the input device(s) 906 to visually output various data described herein. In some implementations, the input device(s) 906 includes one or more touch sensors, the output device(s) 908 includes a display screen, and the touch sensor(s) are integrated with the display screen.

In various implementations, the processor(s) 902 is operably connected to one or more transceivers 910 that transmit and/or receive data over one or more communication networks 912. For example, the transceiver(s) 910 includes a network interface card (NIC), a network adapter, a local area network (LAN) adapter, or a physical, virtual, or logical address to connect to the various external devices and/or systems. In various examples, the transceiver(s) 910 includes any sort of wireless transceivers capable of engaging in wireless communication (e.g., radio frequency (RF) communication). For example, the communication network(s) 912 includes one or more wireless networks that include a 3rd Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 910 includes other wireless modems, such as a modem for engaging in WI-FIR, WIGIG®, WIMAX®, BLUETOOTH®, or infrared communication over the communication network(s) 912.

The device(s) 900 may further include the sequencer 112. In various implementations, the sequencer 112 includes one or more fluidic circuits 914 configured to receive a sample 916 derived from a subject 917. The sequencer 112, in various cases, may be configured to generate data indicative of one or more sequences of nucleic acid molecules (e.g., DNA and/or RNA) present in the sample 916. In various cases, the sequencer 112 introduces one or more reagents 918 to the fluidic circuit(s) 914 in order to prepare for and perform sequencing of the nucleic acid molecules. Further, the sequencer 112 may include one or more sensors 920 configured to measure or otherwise detect detection signals from the fluidic circuit(s) 914, which may be indicative of the sequences of the nucleic acid molecules. According to various implementations, the sensor(s) 920 may further include one or more ADCs. The sequencer 112, in various cases, outputs sequence read data to the processor(s) 902 for additional processing.

### Experimental Example

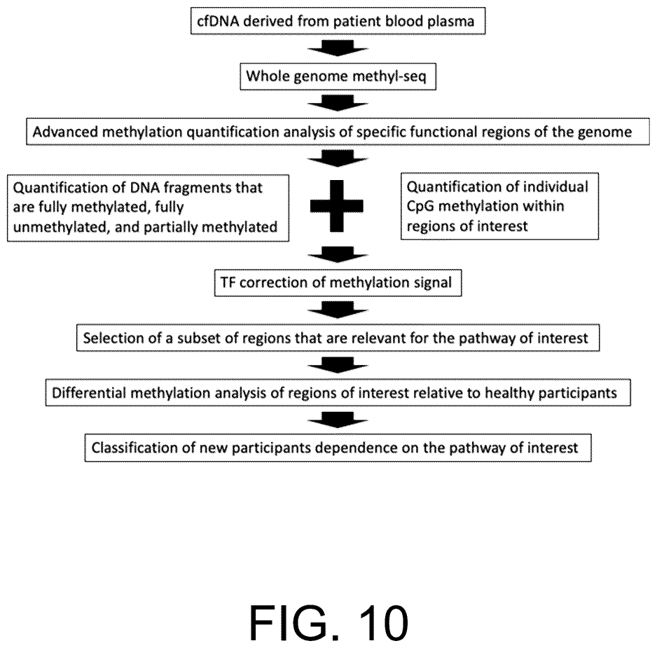

In a particular example, whole genome methyl-seq was performed on cfDNA from patients with a specific disease ontology. FIG. 10 illustrates an example process utilized in this Example. Methylation of specified regions genome-wide (such as promoters, CpG islands) was quantified. This was performed via (a) quantitation of the fraction of DNA fragments that are fully methylated, fully unmethylated, and partially methylated within each particular region of interest genome wide; and (b) individual CpG resolution methylation traces across regions of interest.

The methylation status of each sample was corrected for differential tumor fraction. This was performed by estimating tumor fraction (TF) from the data and taking a weighted average of patient's signal (weighted by TF) and the average in unaffected persons (weighed as 1-TF) in order to estimate the signal in the tumor. For example, if the fraction of fully methylated fragments at a promoter is 90% in a patient and the tumor fraction for the patient is 20% while unaffected people display 100% methylation, then the tumor has 50% signal. This correction gives a normalized ctDNA methylation status, inferring the underlying methylation pattern of the tumor itself.

Rather than proceeding with the genome wide methylation statuses, in this example, the methylation statuses of derived from a subset of the regions of the genome were identified. These regions were potential parts of an expression pathway of interest, either as promoters of the genes in the pathway or as nearby CpG islands. This gives a methylation status specific to the pathways of interest.

Differential methylation analysis and refinement of this pathway-relevant methylation status was performed. This analysis can be unsupervised (e.g., if the groups of patients that respond to the treatment are not known) or supervised (e.g., if the patient responses are known) and includes comparison between healthy and affected participants.

Next, a determination was made of a new patient's tumor's dependence on the pathway of interest based which group their methylation status is assigned to. For example, the nearest neighbor algorithm is applied to normalized ctDNA methylation status for an unknown patient and a group of patients with known tumor pathway status. If the patient's normalized ctDNA methylation status is nearest to patients whose tumors are dependent on the pathway of interest, then the patient is likely to respond to the treatment targeting this pathway. This was performed with hierarchical clustering and principal component analysis (PCA).

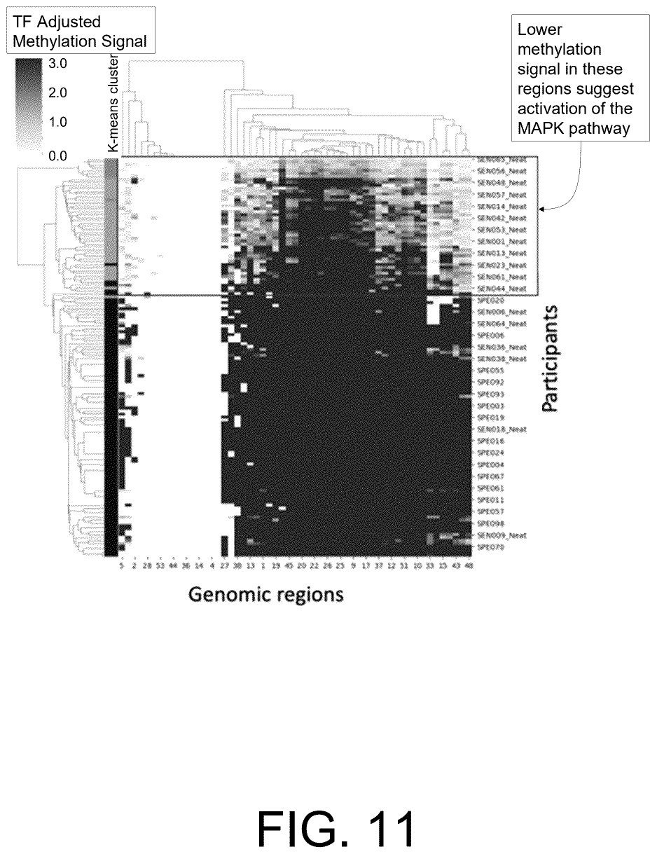

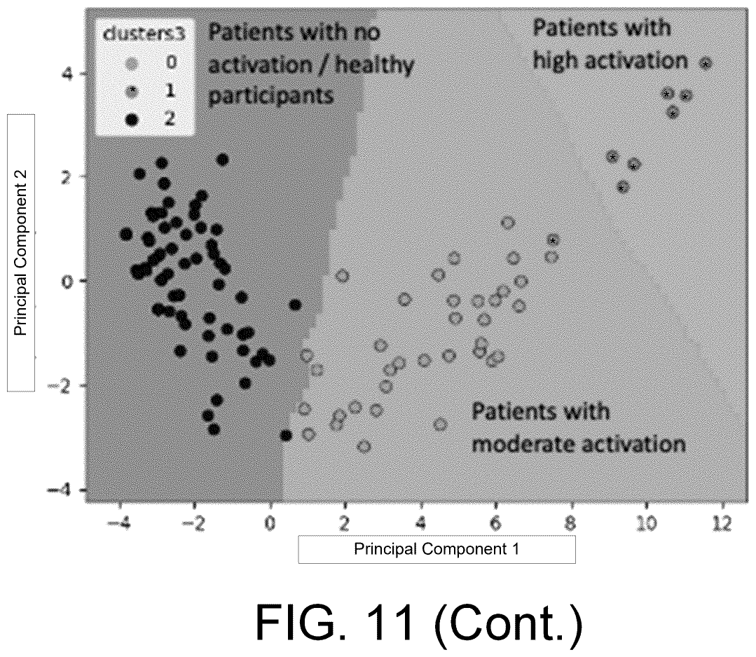

FIG. 11 illustrates example results of an analysis performed on regions of ctDNA related to the mitogen-activated protein kinase (MAPK) signaling pathway. Kirsten rat sarcoma virus (KRAS) signaling is frequently aberrant in cancer, often as a result of KRAS gene mutations. These mutations can lead to activation of the MAPK signaling pathway which promotes cell proliferation. In this example, the methylation statuses of several regions in the cfDNA of several individuals was identified. These regions, for example, include promotors and CpG islands of several genes within this pathway. In this example, a methylation signal (e.g., a metric indicating an amount of methylation) for each region for each participant was determined. Individuals with relatively low methylation signals in the regions indicate that the pathway is active in these patients and they may benefit from therapies that inhibit its activity.

### Example Clauses

The following clauses provide various implementations of the present disclosure.
1: A method, including: providing a plurality of nucleic acid molecules obtained from a sample of a subject, the nucleic acid molecules including cell free DNA (cfDNA); ligating one or more adapters to one or more nucleic acid molecules from the plurality of nucleic acid molecules; amplifying the one or more ligated nucleic acid molecules from the plurality of nucleic acid molecules; capturing all or a subset of the amplified nucleic acid molecules; and sequencing, by a sequencer, all or a subset of the captured nucleic acid molecules to obtain a plurality of sequence reads that represent the captured nucleic acid molecules; receiving, at one or more processors, sequence read data for the plurality of sequence reads; identifying, using the one or more processors, a methylation status of one or more regions in circulating tumor DNA (ctDNA) among the cfDNA based on analyzing the sequence read data; inputting input data including the methylation status of the one or more regions in the ctDNA into at least one model configured to generate a probability that cancer cells of the subject express a predetermined sequence; and generating, using the one or more processors, a report based on the probability that the cancer cells of the subject express the predetermined sequence.
2: The method of clause 1, wherein the sample includes a liquid biopsy sample.
3: The method of clause 1 or 2, wherein the one or more regions include one or more of: at least one gene; at least one promoter; at least one enhancer; or at least one CpG island.
4: The method of any of clauses 1-3, wherein identifying, using the one or more processors, the methylation status of one or more regions in the ctDNA among the cfDNA by analyzing the sequence read data includes: determining a tumor fraction of the cfDNA by analyzing the sequence read data; calculating a correction based on the tumor fraction and a methylation status of the one or more regions in a genome of at least one individual without cancer; determining a methylation status of the one or more regions in the cfDNA; and identifying the methylation status of the one or more regions in the ctDNA based on the methylation status of the one or more regions of the cfDNA and the correction.
5: The method of any of clauses 1-4, wherein the model includes at least one machine learning (ML) model.
6: A method, including: identifying data indicative of cell free DNA (cfDNA) from a sample derived from a subject; identifying, by analyzing the data using one or more processors, a methylation status of one or more regions of circulating tumor DNA (ctDNA) among the cfDNA; inputting, using the one or more processors, input data including the methylation status of the one or more regions into at least one model configured to generate a probability that cancer cells of the subject express a predetermined sequence; and generating, using the one or more processors, a report based on the probability that the cancer cells of the subject express the predetermined sequence.
7: The method of clause 6, wherein the cfDNA includes at least one fragment having a length in a range of about 1 base to about 500 bases.
8: The method of clause 6 or 7, wherein the sample includes a liquid biopsy sample.
9: The method of any of clauses 6-8, wherein the sample includes a blood sample.
10: The method of any of clauses 6-9, wherein the sample includes plasma.
11: The method of any of clauses 6-10, wherein the subject has adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease, intestinal cancer, kidney cancer, larynx cancer, leukemia, liver cancer, lymph node cancer, lymphoma, lung cancer, melanoma, mesothelioma, myeloma, nasopharynx cancer, a neuroblastoma, non-Hodgkin's lymphoma, oral cancer, ovarian cancer, pancreatic cancer, penile cancer, pharynx cancer, prostate cancer, rectal cancer, sarcoma, seminoma, skin cancer, stomach cancer, a teratoma, testicular cancer, thyroid cancer, uterine cancer, vaginal cancer, a vascular tumor, or combinations or metastases thereof.
12: The method of any of clauses 6-11, wherein the data indicative of the cfDNA includes methylation data of the cfDNA.
13: The method of clause 12, further including: generating one or more converted nucleic acid molecules by converting, using at least one enzyme, a portion of cytosines in the one or more nucleic acid molecules into uracils, the one or more nucleic acid molecules including the cfDNA; ligating one or more adapters onto one or more one or more converted nucleic acid molecules; amplifying the one or more ligated nucleic acid molecules; capturing all or a subset of the amplified nucleic acid molecules; generating sequence read data by sequencing, by a sequencer, the captured nucleic acid molecules to obtain a plurality of sequence reads that represent the captured nucleic acid molecules; identifying, among the plurality of sequence reads, methylated cytosines in the cfDNA by comparing the sequence read data to one or more reference sequences; and generating methylation data based on the sequence read data and the identified methylated cytosines.
14: The method of clause 13, wherein the portion of the cytosines in the one or more nucleic acid molecules includes nonmethylated cytosines.
15: The method of clause 14, wherein at least one enzyme includes a bisulfite.
16: The method of clause 15, wherein the bisulfite includes sodium bisulfite.
17: The method of any of clauses 14-16, wherein the one or more converted nucleic acid molecules include one or more nonmethylated cytosines.
18: The method of any of clauses 13-17, wherein the portion of the cytosines in the one or more nucleic acid molecules includes methylated cytosines.
19: The method of clause 18, wherein the at least one enzyme includes one or more of tet methylcytosine dioxygenase 2 (TET2), T4-phage beta-glucosyltransferase (T4-BGT), or apolipoprotein B mRNA editing enzyme catalytic polypeptide (APOBEC).
20: The method of clause 18 or 19, wherein the one or more converted nucleic acid molecules include one or more methylated cytosines.
21: The method of any of clauses 13-20, further including: extracting the one or more nucleic acid molecules from the sample.
22: The method of any of clauses 13-21, wherein the one or more adapters include amplification primers, flow cell adaptor sequences, substrate adapter sequences, or sample index sequences.
23: The method of any of clauses 13-22, wherein the captured nucleic acid molecules are captured from the amplified nucleic acid molecules by hybridization to one or more bait molecules.
24: The method of clause 23, wherein the one or more bait molecules include one or more additional nucleic acid molecules, each of the one or more additional nucleic acid molecules including a region that is complementary to a region of a captured nucleic acid molecule.
25: The method of any of clauses 13-24, wherein amplifying the one or more ligated nucleic acid molecules includes performing a polymerase chain reaction (PCR) amplification technique, a non-PCR amplification technique, or an isothermal amplification technique.
26: The method of any of clauses 13-25, wherein sequencing the captured nucleic acid molecules includes use of a massively parallel sequencing (MPS) technique, whole genome sequencing (WGS), whole exome sequencing, targeted sequencing, direct sequencing, or Sanger sequencing.
27: The method of any of clauses 13-26, wherein sequencing the captured nucleic acid molecules includes next generation sequencing (NGS).
28: The method of clause 27, wherein sequencing the captured nucleic acid molecules is performed by a next generation sequencer.
29: The method of any of clauses 13-28, wherein sequencing the captured nucleic acid molecules includes sequencing-by-synthesis or nanopore sequencing.
30: The method of any of clauses 12-29, further including: generating one or more converted nucleic acid molecules by converting, using at least one enzyme, a portion of cytosines in the one or more nucleic acid molecules into uracils, the one or more nucleic acid molecules including the cfDNA generating ligated molecules by ligating adaptors onto the one or more converted nucleic acid molecules; generating amplified ligated molecules by amplifying the ligated molecules; generating, using the amplified ligated molecules, detection signals; detecting, by at least one sensor, the detection signals; generating sequence read data based on the detection signals; and generating the methylation data based on the sequence read data.
31: The method of clause 30, wherein the detection signals include electrical signals and/or optical signals.
32: The method of clause 30 or 31, wherein generating, using the amplified ligated molecules, the detection signals includes simultaneously: synthesizing, by a polymerase using fluorescently tagged nucleotide triphosphates (NTPs), a synthesized nucleic acid molecule based on one of the amplified ligated molecules, and wherein detecting, by the at least one sensor, the detection signals include: detecting, by at least one optical sensor, optical signals emitted by the fluorescently tagged NTPs upon binding to the synthesized nucleic acid molecule, the optical signals being indicative of at least one sequence of the cfDNA.
33: The method of any of clauses 30-32, wherein generating, using the amplified ligated molecules, the detection signals include simultaneously: directing the amplified ligated molecules through a nanopore extending from a first space to a second space through a substrate, and wherein detecting, by the at least one sensor, the detection signals include: detecting, by sensors disposed in the first space and the second space, an electrical signal over time, the electrical signal being indicative of at least one sequence of the cfDNA.
34: The method of any of clauses 6-33, further including: receiving the sample.
35: The method of clause 34, wherein the sample includes blood, plasma, cerebrospinal fluid, sputum, stool, urine, pleural lavage, lymphatic fluid, or saliva.
36: The method of clause 34 or 35, wherein the sample further includes genomic DNA (gDNA).
37: The method of clause 36, further including: extracting the gDNA from the sample, wherein identifying the data indicative of the cfDNA includes sequencing the gDNA.
38: The method of any of clauses 6-37, further including: identifying a portion of the data indicative of the ctDNA, wherein the input data includes the portion of the data indicative of the ctDNA.
39: The method of clause 38, wherein determining the portion of the data indicative of the ctDNA is based on at least one of: one or more lengths of the sequences of the cfDNA; one or more variants in the sequences of the cfDNA; one or more relative read depths of the cfDNA; or one or more genomic coordinates of the cfDNA.
40: The method of any of clauses 6-39, wherein identifying, by analyzing the data, the methylation status of the one or more regions in the ctDNA among the cfDNA includes: determining a methylation status of the one or more regions in the cfDNA; and identifying the methylation status of the one or more regions in the ctDNA based on the methylation status of the one or more regions in the cfDNA.
41: The method of clause 40, further including: determining a correction based on an amount of the ctDNA in the sample, wherein identifying the methylation status of the one or more regions in the ctDNA is further based on the correction.
42: The method of clause 41, wherein determining the correction based on the amount of the ctDNA in the sample includes: determining a tumor fraction of the sample.
43: The method of clause 42, wherein determining the tumor fraction of the sample includes: acquiring a value for a target variable associated with a subgenomic interval in the sample; determining, from the target variable, a certainty metric; accessing a determined relationship between a stored certainty metric and a stored tumor fraction; and determining, with reference to the certainty metric and the determined relationship, the tumor fraction of the sample.
44: The method of clause 42 or 43, wherein determining the correction based on the amount of the ctDNA in the sample is further based on a methylation status of the one or more regions in a genome of at least one individual without cancer.
45: The method of any of clauses 40-44, wherein the methylation status of the one or more regions in the sample is calculated from the ctDNA methylation status based on the following equation:

$${m}_{t} = \frac{{m}_{p} - {m}_{0}}{f} + {m}_{0} ,$$

wherein mₜ includes the methylation status as a fraction of the patient's tumor's DNA in one or more regions, mₚ includes a methylation status of the one or more regions in the cfDNA, f includes a tumor fraction of the sample, and m₀ includes a methylation status of the one or more regions in a genome of at least one individual without cancer.

46: The method of any of clauses 6-45, wherein the methylation status includes an amount of methylated cytosines in the one or more regions.
47: The method of clause 46, wherein the methylation status includes at least one of: a percentage of methylated cytosines in the one or more regions; a number of methylated cytosines in the one or more regions; or a density of methylated cytosines in the one or more regions.
48: The method of clause 46 or 47, wherein the methylation status includes whether the amount of methylated cytosines in the one or more regions is above a first threshold and/or below a second threshold.
49: The method of any of clauses 6-48, wherein the one or more regions includes at least a portion of a gene.
50: The method of clause 49, wherein the predetermined sequence includes a gene.
51: The method of any of clauses 6-50, wherein the one or more regions include at least a portion of a promoter.
52: The method of clause 51, wherein the promoter is operably linked to a gene.
53: The method of clause 52, wherein the predetermined sequence includes the gene operably linked to the promoter.
54: The method of any of clauses 6-53, wherein the one or more regions include at least a portion of an enhancer.
55: The method of clause 54, wherein the predetermined sequence includes a gene operably linked to the enhancer.
56: The method of any of clauses 6-55, wherein the one or more regions include at least a portion of a CpG island.
57: The method of any of clause 56, wherein the CpG island is within a threshold distance of a gene in a reference genome, and wherein the predetermined sequence includes the gene.
58: The method of any of clauses 6-57, wherein the model includes at least one machine learning (ML) model.
59: The method of clause 58, wherein the at least one ML model includes at least one of a neural network, a nearest-neighbor model, a regression analysis model, a clustering model, principal component analysis model, a gradient boosting model, or a random forest.
60: The method of clause 58 or 59, further including: training the ML model by optimizing parameters of the ML model based on training data, the training data including example methylation states of the one or more regions identified from example samples of a population.
61: The method of clause 60, wherein the population omits the subject.
62: The method of clause 60 or 61, wherein the population includes at least one first individual and at least one second individual, the at least one first individual including cancer cells substantially expressing the predetermined sequence the at least one second individual including cancer cells that do not express the predetermined sequence.
63: The method of any of clauses 60-62, wherein: the training data further includes labels indicating whether the example samples are obtained from at least one individual having cancer cells expressing the predetermined sequence, and wherein training the ML model includes identifying, using supervised ML based on pairs of the labels and corresponding instances of the example methylation states, predictive attributes of the example methylation states that are indicative of the labels.
64: The method of clause 63, wherein training the ML model includes configuring the ML model to, based on the input data: identify instances of the predictive attributes associated with the methylation status of the one or more regions in the ctDNA; and generate the probability that the cancer cells of the subject express the predetermined sequence is based on the instances of the predictive attributes.
65: The method of any of clauses 60-64, wherein training the ML model includes identifying, via unsupervised ML, a plurality of clusters of the example methylation states that are indicative of whether the clusters are in a uniform state based on the expression of the predetermined sequence.
66: The method of clause 65, wherein training the ML model includes configuring the ML model to, based on the input data: identify a cluster, of the plurality of clusters, associated with the methylation states of the one or more regions in the ctDNA; and generate the probability that the cancer cells of the subject express the predetermined sequence based on the cluster associated with the methylation states.
67: The method of clause 66, wherein the ML model is configured to generate the probability that the cancer cells of the subject express the predetermined sequence based on at least one distance between the cluster and the methylation status of the ctDNA in a cluster space.
68: The method of any of clauses 60-67, wherein the at least one ML model includes: a first ML model configured to generate a first probability that the cancer cells of the subject express a first gene; and a second ML model configured to generate a second probability that the cancer cells of the subject express a second gene, wherein the probability that the cancer cells of the subject express the predetermined sequence is based on the first probability and the second probability.
69: The method of clause 68, further including: identifying example methylation statuses of example ctDNA in example samples obtained from a population; identifying first labels indicating whether the population has cancer cells expressing the first gene; identifying second labels indicating whether the population has cancer cells expressing the second gene; training the first ML model based on first training data including: the example methylation statuses; and the first labels; and training the second ML model based on second training data including: the example methylation statuses; and the second labels.
70: The method of any of clauses 6-69, wherein the predetermined sequence includes one or more genes.
71: The method of clause 70, wherein the one or more genes are associated with resistance to an anticancer therapy.
72: The method of clause 71, wherein the anticancer therapy includes at least one of surgery, a chemotherapy, a radiotherapy, or an immunotherapy.
73: The method of clause 71 or 72, wherein the anticancer therapy includes an immunotherapy.
74: The method of any of clauses 70-73, wherein the one or more genes are associated with responsiveness to an anticancer therapy.
75: The method of clause 74, wherein the anticancer therapy includes at least one of surgery, a chemotherapy, a radiotherapy, or an immunotherapy.
76: The method of clause 74 or 75, wherein the anticancer therapy includes an immunotherapy.
77: The method of any of clauses 70-76, wherein the one or more genes include KRAS.
78: The method of any of clauses 6-77, wherein generating the report based on the at least one probability that the cancer cells of the subject express the predetermined sequence includes: determining that the probability exceeds a threshold; and generating the report to indicate that the cancer cells of the subject are predicted to express the predetermined sequence.
79: The method of clause 78, wherein generating the report to indicate that the cancer cells of the subject are predicted to express the predetermined sequence includes generating the report to indicate an instruction to perform a follow-up test on the subject.
80: The method of clause 79, wherein the follow-up test includes obtaining a tissue biopsy of a tumor of the subject.
81: The method of clause 80, wherein the follow-up test includes at least one of: a histological study; whole transcriptome sequencing; cfRNA sequencing; whole exome sequencing; whole genome sequencing a cancer hotspot panel test; a DNA methylation test; a DNA fragmentation test; an RNA fragmentation test; a microsatellite instability (MSI) test; a tumor mutational burden (TMB) test; or a viral status test.
82: The method of any of clauses 79-81, further including: identifying additional data indicating results of the follow-up test; determining at least one updated probability that the cancer cells of the subject express the predetermined sequence; generating an updated report based on the at least one updated probability; and outputting the updated report.
83: The method of any of clauses 78-82, wherein generating the report to indicate that the cancer cells of the subject are predicted to express the predetermined sequence includes generating the report to indicate a recommendation to administer a predetermined therapy to the subject.
84: The method of clause 83, wherein the predetermined therapy includes at least one of surgery, a chemotherapy, a radiotherapy, or an immunotherapy.
85: The method of any of clauses 6-84, further including: generating a genomic profile of the subject, the report including the genomic profile.
86: The method of clause 85, wherein the genomic profile includes results from at least one of: a histological study; whole transcriptome sequencing; cfRNA sequencing; whole exome sequencing; whole genome sequencing a cancer hotspot panel test; a DNA methylation test; a DNA fragmentation test; an RNA fragmentation test. a microsatellite instability (MSI) test; a tumor mutational burden (TMB) test; or a viral status test.
87: The method of clause 85 or 86, wherein the genomic profile of the subject includes: results from a nucleic acid sequencing-based test.
88: The method of any of clauses 85-87, further including: selecting, based on the genomic profile and/or the probability that the cancer cells of the subject express the predetermined sequence, an anticancer agent for administration to the subject.
89: The method of clause 88, further including: administering the anticancer agent to the subject.
90: The method of any of clauses 85-89, further including: applying, based on the genomic profile, an anticancer therapy to the subject.
91: The method of clause 90, wherein the anticancer therapy includes at least one of a surgery, a chemotherapy, a radiotherapy, or an immunotherapy.
92: The method of any of clauses 85-91, further including: identifying, based on the probability that the cancer cells of the subject express the predetermined sequence, a suggested treatment decision for the subject, the report including the suggested treatment decision.
93: The method of clause 92, wherein the suggested treatment decision includes at least one of a surgery, a chemotherapy, a radiotherapy, or an immunotherapy.
94: The method of clause 92 or 93, wherein the suggested treatment decision includes a dosage of one or more therapeutic agents predicted to treat the cancer cells.
95: The method of any of clauses 6-94, further including: outputting the report.
96: The method of clause 95, wherein outputting the report includes: transmitting data indicating the report to an external device.
97: The method of clause 96, wherein the external device is associated with the subject or a healthcare provider.
98: The method of clause 96 or 97, wherein the data indicating the report is transmitted over one or more communication networks.
99: The method of any of clauses 96-98, wherein the data indicating the report is transmitted over a peer-to-peer connection.
100: The method of any of clauses 6-99, wherein outputting the report includes: visually presenting, by a display, the report.
101: The method of any of clauses 6-100, further including: determining, based on the at least one probability that the cancer cells of the subject express the predetermined sequence, whether the subject is eligible for a clinical trial, wherein the report indicates whether the subject is eligible for the clinical trial.
102: A system, including: at least one processor; and memory storing instructions that, when executed by the at least one processor, cause the at least one processor to perform operations including: identifying data indicative of cell free DNA (cfDNA) from a sample derived from a subject; identifying, by analyzing the data, a methylation status of one or more regions of circulating tumor DNA (ctDNA) among the cfDNA; inputting input data including the methylation status of the one or more regions into at least one model configured to generate a probability that cancer cells of the subject express a predetermined sequence; and generating a report based on the probability that the cancer cells of the subject express the predetermined sequence.
103: The system of clause 102, further including: a sequencer configured to generate the data by sequencing the cfDNA.
104: The system of clause 103, further including: a transceiver configured to receive a communication signal encoding the data.
105: The system of clause 103 or 104, further including: a transceiver configured to transmit, to an external device, a communication signal encoding the report.
106: The system of any of clauses 103-105, further including: a display configured to visually present the report.
107: A non-transitory computer readable medium storing instructions for performing operations including: identifying data indicative of cell free DNA (cfDNA) from a sample derived from a subject; identifying, by analyzing the data, a methylation status of one or more regions of circulating tumor DNA (ctDNA) among the cfDNA; inputting input data including the methylation status of the one or more regions into at least one model configured to generate a probability that cancer cells of the subject express a predetermined sequence; and generating a report based on the probability that the cancer cells of the subject express the predetermined sequence.

### CONCLUSION

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e., denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Tumor mutational burden (TMB) is a measure of the number of mutations carried by tumor cells. By comparing DNA sequences from a patient's healthy tissues and tumor cells, the number of acquired somatic mutations present in tumors, but not in normal tissues, can be determined.

In certain examples, “tumor mutational burden” or “TMB” refers to the number of somatic mutations in a tumor's genome and/or the number of somatic mutations per area of the tumor's genome. In some embodiments, TMB, as used herein, refers to the number of somatic mutations per megabase (Mb) of DNA sequenced. In some embodiments, germline (inherited) variants are excluded when determining TMB, given that the immune system has a higher likelihood of recognizing these as self.

Microsatellites are highly polymorphic DNA-repeat regions. In certain examples, “microsatellite” refers to a repetitive nucleic acid having repeat units of less than about 10 base pairs or nucleotides in length. In certain examples, a microsatellite refers to a tract of tandemly repeated (i.e. adjacent) DNA motifs ranging from one to six or up to ten nucleotides, with each motif repeated 5 to 50 repeated times. “Microsatellite instability” refers to genetic instability in the microsatellite regions. Cancer patients with microsatellite instability classified as being high (MSI-H or MSI-High) frequently exhibit an accumulation of somatic mutations in tumor cells that leads to a range of molecular and biological changes including high tumor mutational burden, increased expression of neoantigens and abundant tumor-infiltrating lymphocytes. Chang et al. “Microsatellite Instability: A Predictive Biomarker for Cancer Immunotherapy,” Appl Immunohistochem Mol Morphol, 26 (2): e15-e21 (2018). These changes have been linked to increased sensitivity to checkpoint inhibitor drugs, such as pembrolizumab, which is used to treat advanced melanoma, head and neck squamous cell carcinoma, non-small cell lung cancer (NSCLC), and classical Hodgkin lymphoma.

A viral status test refers to a test that identifies the presence of viral RNA or DNA in a subject. The test can identify viral load and/or viral identity. For example, the viral status test can identify the presence of viral RNA or DNA associated with the occurrence of certain cancers. Examples of such viruses include Hepatitis B Virus (HBV) and Hepatitis C Virus (HCV), Kaposi Sarcoma-Associated Herpesvirus (KSHV), Merkel Cell Polyomavirus (MCV), Human Papillomavirus (HPV), Human Immunodeficiency Virus Type 1 (HIV-1, or HIV), Human T-Cell Lymphotropic Virus Type 1 (HTLV-1), and Epstein-Barr Virus (EBV).

Cancer “hotspot” mutations give rise to oncological outcomes. PhyloP, SIFT, Grantham, COSMIC and PolyPhen-2 are in silico tools that can be used to assess pathogenicity of identified variants. Exemplary hotspot genes and mutations include EGFR exon 19 activating mutation, EGFR exon 19 deletion, EGFR exon 19 insertion, EGFR exon 19 sensitizing mutation, EGFR exon 20 activation mutation, EGFR exon 20 insertion, EGFR G719 mutation, EGFR L858R mutation, EGFR L861 mutation, EGFR S768 mutation, EGFR T790M mutation, KIT activating mutation, KRAS activating mutation, MET activating mutation, NRAS activating mutation, PMS2 promoter mutations, among many others. Hotspot mutations also occur in the following genes: AKT2, BRCA1, BRCA2, ERC1, NSD1, POLH, PPM1G, PTEN, RAD18, RAD51, RAD51B, RB1, TERT, TP53, TP53Bp1, ALK, ARMT1, ATAD5, ATG7, ATIC, AXL, BIRC6, BRD3, BRD4, CAPRIN1, CCAR2, CCDC6, CDK5RAP2, CHD9, CIT, CTNNB1, CUL1, EBF1, EIF3E, HIP1, HMGA2, IRF2BP2, NOTCH1, NOTCH4, NPM1, OFD1, TACC1, TACC3, TERF2, TMEM106B, UBE2L3, USP10, WRDR48, YAP1, ZEB2, and ZMYND8.

A “DNA methylation test” refers to an assay, which can be commercially available, for distinguishing methylated versus unmethylated cytosine loci in DNA. Techniques for measuring cytosine methylation include bisulfite-based methylation assays. The addition of bisulfite to DNA results in the methylation of unmethylated cytosine and its ultimate conversion to the nucleotide uracil. Uracil has similar binding properties to thiamine in the DNA sequence. Previously methylated cytosine does not undergo similar chemical conversion on exposure to bisulfite. Bisulfite assays can thus be used to discriminate previously methylated versus unmethylated cytosine.

An exemplary quantitative methylation detection assay combines bisulfite treatment and restriction analysis COBRA, which uses methylation sensitive restriction endonucleases, gel electrophoresis, and detection based on labeled hybridization probes. (Ziong and Laird, Nucleic Acid Res. 1997 25; 2532-4). Another exemplary detection assay is the methylation specific polymerase chain reaction PCR (MSPCR) for amplification of DNA segments of interest. This assay can be performed after sodium bisulfite conversion of cytosine and uses methylation sensitive probes. Other detection assays include the Quantitative Methylation (QM) assay, which combines PCR amplification with fluorescent probes designed to bind to putative methylation sites; MethyLight™ (Qiagen, Redwood City, CA) a quantitative methylation detection assay that uses fluorescence based PCR (Eads, et al., Cancer Res. 1999; 59:2302-2306); and Ms-SNuPE, a quantitative technique for determining differences in methylation levels in CpG sites. As with other techniques, Ms-SNuPE also requires bisulfite treatment to be performed first, leading to the conversion of unmethylated cytosine to uracil while methyl cytosine is unaffected. PCR primers specific for bisulfite converted DNA are then used to amplify the target sequence of interest. The amplified PCR product is isolated and used to quantitate the methylation status of the CpG site of interest. (Gonzalgo and Jones Nuclei Acids Res 1997; 25:252-31).

In particular embodiments, pyrosequencing can be used to detect marker methylation. Pyrosequencing is a method of DNA sequencing that relies on detection of the release of pyrophosphates as DNA is synthesized (and is therefore a “sequencing by synthesis” technique). To assess methylation by pyrosequencing, a DNA sample can be incubated with sodium bisulfite, converting unmethylated cytosine to uracil. The presence of uracil will result in thymine incorporation during PCR amplification. Therefore, sequencing results that include thymine at a nucleotide position that is known to encode cytosine can be interpreted as unmethylated sites. In contrast cytosines present in the sequencing results indicate that the site was methylated in the original DNA sample, because methylation protects cytosine from conversion to uracil upon treatment. Bisulfite treatment can also be performed on control samples with known methylation patterns, to reduce or eliminate false positive results. Commercially available pyrosequencing machines include Pyro Mark Q96 (Qiagen, Hilden, Germany). For more details on methods to use pyrosequencing for measurement of methylation, see Delaney et al. Methods Mol Biol. 2015 1343:249-264. Pyrosequencing is especially useful for detecting methylation in the CpG sites within genes.

In particular embodiments, a protein marker is detected by contacting a sample with reagents (e.g., antibodies), generating complexes of reagent and marker(s), and detecting the complexes. Particular embodiments for detecting and measuring protein levels can use methods including agglutination, chemiluminescence, electro-chemiluminescence (ECL), enzyme-linked immunoassays (ELISA), immunoassay, immunoblotting, immunodiffusion, immunoelectrophoresis, immunofluorescence, immunohistochemistry, immunoprecipitation, mass-spectrometry, and western blot. See also, e.g., E. Maggio, Enzyme-Immunoassay (1980), CRC Press, Inc., Boca Raton, Fla; and U.S. Pat. Nos. 4,727,022; 4,659,678; 4,376,110; 4,275,149; 4,233,402; and 4,230,797.

Read depth refers to the number of times that a specific genomic site is sequenced during a sequencing run.

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A method, comprising:
identifying data indicative of cell free DNA (cfDNA) from a sample derived from a subject;
identifying, by analyzing the data using one or more processors, a methylation status of one or more regions of circulating tumor DNA (ctDNA) among the cfDNA;
inputting, using the one or more processors, input data comprising the methylation status of the one or more regions into at least one model configured to generate a probability that cancer cells of the subject express a predetermined sequence; and
generating, using the one or more processors, a report based on the probability that the cancer cells of the subject express the predetermined sequence.

2. The method of claim 1, wherein the cfDNA comprises at least one fragment having a length in a range of about 1 base to about 500 bases.

3. The method of claim 1, wherein the sample comprises a liquid biopsy sample.

4. The method of claim 1, wherein the data indicative of the cfDNA comprises methylation data of the cfDNA, and wherein the method further comprises:
generating one or more converted nucleic acid molecules by converting, using at least one enzyme, a portion of cytosines in the one or more nucleic acid molecules into uracils, the one or more nucleic acid molecules comprising the cfDNA;
ligating one or more adapters onto one or more one or more converted nucleic acid molecules;
amplifying the one or more ligated nucleic acid molecules;
capturing all or a subset of the amplified nucleic acid molecules;
generating sequence read data by sequencing, by a sequencer, the captured nucleic acid molecules to obtain a plurality of sequence reads that represent the captured nucleic acid molecules;
identifying, among the plurality of sequence reads, methylated cytosines in the cfDNA by comparing the sequence read data to one or more reference sequences; and
generating methylation data based on the sequence read data and the identified methylated cytosines.

5. The method of claim 4, wherein the portion of the cytosines in the one or more nucleic acid molecules comprises nonmethylated cytosines, wherein at least one enzyme comprises a bisulfite, and wherein the one or more converted nucleic acid molecules comprise one or more nonmethylated cytosines.

6. The method of claim 4, wherein the portion of the cytosines in the one or more nucleic acid molecules comprises methylated cytosines, wherein the at least one enzyme comprises one or more of tet methylcytosine dioxygenase 2 (TET2), T4-phage beta-glucosyltransferase (T4-BGT), or apolipoprotein B mRNA editing enzyme catalytic polypeptide (APOBEC), and wherein the one or more converted nucleic acid molecules comprise one or more methylated cytosines.

7. The method of claim 1, further comprising:
identifying a portion of the data indicative of the ctDNA,
wherein the input data comprises the portion of the data indicative of the ctDNA, and
wherein determining the portion of the data indicative of the ctDNA is based on at least one of:
one or more lengths of sequences of the cfDNA,
one or more variants in the sequences of the cfDNA,
one or more relative read depths of the cfDNA, or
one or more genomic coordinates of the cfDNA.

8. The method of claim 1, wherein identifying, by analyzing the data, the methylation status of the one or more regions in the ctDNA among the cfDNA comprises:
determining the methylation status of the one or more regions in the cfDNA; and
identifying the methylation status of the one or more regions in the ctDNA based on the methylation status of the one or more regions in the cfDNA.

9. The method of claim 8, further comprising:
determining a correction based on an amount of the ctDNA in the sample,
wherein identifying the methylation status of the one or more regions in the ctDNA is further based on the correction, and
wherein determining the correction based on the amount of the ctDNA in the sample comprises:
determining a tumor fraction of the sample.

10. The method of claim 8, wherein the methylation status of the one or more regions in the sample is calculated from the ctDNA methylation status based on the following equation:
$${m}_{t} = \frac{{m}_{p} - {m}_{0}}{f} + {m}_{0} ,$$
wherein mₜ comprises the methylation status as a fraction of the subject's tumor's DNA in one or more regions, mₚ comprises a methylation status of the one or more regions in the cfDNA, f comprises a tumor fraction of the sample, and m₀ comprises the methylation status of the one or more regions in a genome of at least one individual without cancer.

11. The method of claim 1, wherein the methylation status comprises an amount of methylated cytosines in the one or more regions, and
wherein the methylation status comprises at least one of:
a percentage of methylated cytosines in the one or more regions,
a number of methylated cytosines in the one or more regions, or
a density of methylated cytosines in the one or more regions.

12. The method of claim 1, wherein the one or more regions comprises:
at least a portion of a gene,
the gene,
at least a portion of a promoter operably linked to the gene,
at least a portion of an enhancer operably linked to the gene, or
at least a portion of a CpG island within a threshold distance of the gene.

13. The method of claim 1, wherein the model comprises at least one machine learning (ML) model, and wherein the at least one ML model comprises at least one of a neural network, a nearest-neighbor model, a regression analysis model, a clustering model, principal component analysis model, a gradient boosting model, or a random forest.

14. The method of claim 1, wherein the predetermined sequence comprises one or more genes.

15. The method of claim 14, wherein the one or more genes comprise KRAS.

16. The method of claim 1, wherein generating the report based on the at least one probability that the cancer cells of the subject express the predetermined sequence comprises:
determining that the probability exceeds a threshold; and
generating the report to indicate that the cancer cells of the subject are predicted to express the predetermined sequence.

17. A method, comprising:
providing a plurality of nucleic acid molecules obtained from a sample of a subject, the nucleic acid molecules comprising cell free DNA (cfDNA);
ligating one or more adapters to one or more nucleic acid molecules from the plurality of nucleic acid molecules;
amplifying the one or more ligated nucleic acid molecules from the plurality of nucleic acid molecules;
capturing all or a subset of the amplified nucleic acid molecules;
sequencing, by a sequencer, all or a subset of the captured nucleic acid molecules to obtain a plurality of sequence reads that represent the captured nucleic acid molecules;
receiving, at one or more processors, sequence read data for the plurality of sequence reads;
identifying, using the one or more processors, a methylation status of one or more regions in circulating tumor DNA (ctDNA) among the cfDNA based on analyzing the sequence read data;
inputting input data comprising the methylation status of the one or more regions in the ctDNA into at least one model configured to generate a probability that cancer cells of the subject express a predetermined sequence; and
generating, using the one or more processors, a report based on the probability that the cancer cells of the subject express the predetermined sequence.

18. The method of claim 17, wherein the sample comprises a liquid biopsy sample.

19. The method of claim 17, wherein the one or more regions comprise one or more of:
at least one gene,
at least one promoter,
at least one enhancer, or
at least one CpG island.

20. The method of claim 17, wherein identifying, using the one or more processors, the methylation status of one or more regions in the ctDNA among the cfDNA by analyzing the sequence read data comprises:
determining a tumor fraction of the cfDNA by analyzing the sequence read data;
calculating a correction based on the tumor fraction and the methylation status of the one or more regions in a genome of at least one individual without cancer;
determining the methylation status of the one or more regions in the cfDNA; and
identifying the methylation status of the one or more regions in the ctDNA based on the methylation status of the one or more regions of the cfDNA and the correction.
